SEQUENCE LISTING (43 sequences)

SEQ ID NO: 1 - DNA, 348 bp, Murine
  Murine
caggtccaactgcagcagcctggggttgagcttgtgatgcctggggcttcagtgaagctgtcctgcaaggcttctggctacaccttcaccagctactggatgcactgggtgaagcagaggcctggacaaggccttgagtggatcggcgagattgatccttctgatagtaatactaactacaatcaaaagttcaagggcaaggccacattgactttagacaaatcctccagcacagcctacatgcagctcaccagcctgacatctgaggactcagcggtctattactgtgcaagagaactactgcatgcggtctattggggccaaggcaccactctcacagtctcctca

SEQ ID NO: 2 - DNA, 324 bp, Murine
  Murine
gacatccagatgacacagtctccatcctcactgtctgcatctctggggggcaaagtcaccatcacttgcacggcaagccaagacattcacaaatatatatcttggttccaacataagcctggaaaaggtcctagactgctcatacattacacatctacattacagccaggcatcccatcaaggttcagtgggagtgggtctgggagagattattccttcagcatcagcaacctggagcctgaagatgttgcaacttattattgtctacaatatgataatctgtggacgttcggtggaggcaccaagctggatatcaaacgggct

SEQ ID NO: 3 - protein, 116 aa, Murine
  Murine
QVQLQQPGVELVMPGASVKLSCKASGYTFTSYWMHWVKQRPGQGLEWIGEIDPSDSNTNYNQKFKGKATLTLDKSSSTAYMQLTSLTSEDSAVYYCARELLHAVYWGQGTTLTVSS

SEQ ID NO: 4 - protein, 108 aa, Murine
  Murine
DIQMTQSPSSLSASLGGKVTITCTASQDIHKYISWFQHKPGKGPRLLIHYTSTLQPGIPSRFSGSGSGRDYSFSISNLEPEDVATYYCLQYDNLWTFGGGTKLDIKRA

SEQ ID NO: 5 - protein, 5 aa, Murine
  Murine
SYWMH

SEQ ID NO: 6 - protein, 17 aa, Murine
  Murine
EIDPSDSNTNYNQKFKG

SEQ ID NO: 7 - protein, 7 aa, Murine
  Murine
ELLHAVY

SEQ ID NO: 8 - protein, 11 aa, Murine
  Murine
TASQDIHKYIS

SEQ ID NO: 9 - protein, 6 aa, Murine
  Murine
TSTLQP

SEQ ID NO: 10 - protein, 8 aa, Murine
  Murine
LQYDNLWT

SEQ ID NO: 11 - DNA, 15 bp, Murine
  Murine
agctactggatgcac

SEQ ID NO: 12 - DNA, 51 bp, Murine
  Murine
gagattgatccttctgatagtaatactaactacaatcaaaagttcaagggc

SEQ ID NO: 13 - DNA, 21 bp, Murine
  Murine
gaactactgcatgcggtctat

SEQ ID NO: 14 - DNA, 33 bp, Murine
  Murine
acggcaagccaagacattcacaaatatatatct

SEQ ID NO: 15 - DNA, 18 bp, Murine
  Murine
acatctacattacagcca

SEQ ID NO: 16 - DNA, 24 bp, Murine
  Murine
ctacaatatgataatctgtggacg

SEQ ID NO: 17 - DNA, 348 bp, Murine
  Chimeric
caggtgcagctggtgcagagcggcgccgaggtgaagaagcccggcgccagcgtgaaagtgagctgcaaggccagcggctacaccttcaccagctactggatgcactgggtcaggcaggctcccggccagggcctggagtggatgggcgagatcgaccccagcgacagcaacaccaactacaaccagaagttcaagggcagggtgaccatgaccagggacaccagcatcagcaccgcctacatggaactgagcaggctgaggtccgacgacaccgccgtgtactattgcgccagggaactcctgcacgccgtgtactgggggcagggaacactagtgaccgtgtccagc

SEQ ID NO: 18 - DNA, 348 bp, Artificial Sequence
  Chimeric
caggtgcagctggtgcagagcggcgccgaggtgaagaagcccggcgccagcgtgaaagtgagctgcaaggccagcggctacaccttcaccagctactggatgcactgggtcaggcaggctcccggccagggcctggagtggatgggcgagatcgaccccagcgacagcaacaccaactacaaccagaagttcaagggcaaggtgaccatgaccagggacaccagcatcagcaccgcctacatggaactgagcaggctgaggtccgacgacaccgccgtgtactattgcgccagggaactcctgcacgccgtgtactgggggcagggaacactagtgaccgtgtccagc

SEQ ID NO: 19 - DNA, 321 bp, Artificial Sequence
  Chimeric
gacatccagatgacccagtctcccagcagcctgagcgccagcgtgggcgacagggtgaccattacctgcaccgccagccaggacatccacaagtacatctcctggtaccagcagaagcccggcaaggcccccaagctgctgatctactacactagcaccctgcagcccggcgtcccttcaaggttcagcggaagcggcagcggcaccgacttcaccttcaccatcagcagcctgcagcccgaggatatcgccacctactactgcctgcagtacgacaacctctggaccttcggccagggcaccaaagtggagatcaagcgt

SEQ ID NO: 20 - DNA, 321 bp, Artificial Sequence
  Chimeric
gacatccagatgacccagtctcccagcagcctgagcgccagcgtgggcgacagggtgaccattacctgcaccgccagccaggacatccacaagtacatctcctggtaccagcagaagcccggcaaggcccccaagctgctgatccactacactagcaccctgcagcccggcgtcccttcaaggttcagcggaagcggcagcggcaccgacttcaccttcaccatcagcagcctgcagcccgaggatatcgccacctactactgcctgcagtacgacaacctctggaccttcggccagggcaccaaagtggagatcaagcgt

SEQ ID NO: 21 - DNA, 321 bp, Artificial Sequence
  Chimeric
gacatccagatgacccagtctcccagcagcctgagcgccagcgtgggcgacagggtgaccattacctgcaccgccagccaggacatccacaagtacatctcctggtaccagcagaagcccggcaaggcccccaagctgctgatccactacactagcaccctgcagcccggcgtcccttcaaggttcagcggaagcggcagcggcaccgactacaccttcaccatcagcagcctgcagcccgaggatatcgccacctactactgcctgcagtacgacaacctctggaccttcggccagggcaccaaagtggagatcaagcgt

SEQ ID NO: 22 - protein, 116 aa, Artificial Sequence
  Chimeric
QVQLVQSGAEVKKPGASVKVSCKASGYTFTSYWMHWVRQAPGQGLEWMGEIDPSDSNTNYNQKFKGRVTMTRDTSISTAYMELSRLRSDDTAVYYCARELLHAVYWGQGTLVTVSS

SEQ ID NO: 23 - protein, 116 aa, Artificial Sequence
  Chimeric
QVQLVQSGAEVKKPGASVKVSCKASGYTFTSYWMHWVRQAPGQGLEWMGEIDPSDSNTNYNQKFKGKVTMTRDTSISTAYMELSRLRSDDTAVYYCARELLHAVYWGQGTLVTVSS

SEQ ID NO: 24 - protein, 107 aa, Artificial Sequence
  Chimeric
DIQMTQSPSSLSASVGDRVTITCTASQDIHKYISWYQQKPGKAPKLLIYDTSTLQPGVPSRFSGSGSGTDFTFTISSLQPEDIATYYCLQYDNLWTFGQGTKVEIKR

SEQ ID NO: 25 - protein, 107 aa, Artificial Sequence
  Chimeric
DIQMTQSPSSLSASVGDRVTITCTASQDIHKYISWYQQKPGKAPKLLIHYTSTLQPGVPSRFSGSGSGTDFTFTISSLQPEDIATYYCLQYDNLWTFGQGTKVEIKR

SEQ ID NO: 26 - protein, 107 aa, Artificial Sequence
  Chimeric
DIQMTQSPSSLSASVGDRVTITCTASQDIHKYISWYQQKPGKAPKLLIHYTSTLQPGVPSRFSGSGSGTDYTFTISSLQPEDIATYYCLQYDNLWTFGQGTKVEIKR

SEQ ID NO: 27 - protein, 446 aa, Artificial Sequence
  Chimeric
QVQLQQPGVELVMPGASVKLSCKASGYTFTSYWMHWVKQRPGQGLEWIGEIDPSDSNTNYNQKFKGKATLTLDKSSSTAYMQLTSLTSEDSAVYYCARELLHAVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 213 aa, Artificial Sequence
  Chimeric
DIQMTQSPSSLSASLGGKVTITCTASQDIHKYISWFQHKPGKGPRLLIHYTSTLQPGIPSRFSGSGSGRDYSFSISNLEPEDVATYYCLQYDNLWTFGGGTKLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - DNA, 1341 bp, Artificial Sequence
  Chimeric
caggtccaactgcagcagcctggggttgagcttgtgatgcctggggcttcagtgaagctgtcctgcaaggcttctggctacaccttcaccagctactggatgcactgggtgaagcagaggcctggacaaggccttgagtggatcggcgagattgatccttctgatagtaatactaactacaatcaaaagttcaagggcaaggccacattgactttagacaaatcctccagcacagcctacatgcagctcaccagcctgacatctgaggactcagcggtctattactgtgcaagagaactactgcatgcggtctattggggccaaggcacactagtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 30 - DNA, 642 bp, Artificial Sequence
  Chimeric
gacatccagatgacacagtctccatcctcactgtctgcatctctggggggcaaagtcaccatcacttgcacggcaagccaagacattcacaaatatatatcttggttccaacataagcctggaaaaggtcctagactgctcatacattacacatctacattacagccaggcatcccatcaaggttcagtgggagtgggtctgggagagattattccttcagcatcagcaacctggagcctgaagatgttgcaacttattattgtctacaatatgataatctgtggacgttcggtggaggcaccaagctggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggacaacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 31 - protein, 12 aa, Artificial Sequence
  Homeo sapiens
KTYSKPFHPKFI

SEQ ID NO: 32 - DNA, 1341 bp, Artificial Sequence
  Homeo sapiens
caggtgcagctggtgcagagcggcgccgaggtgaagaagcccggcgccagcgtgaaagtgagctgcaaggccagcggctacaccttcaccagctactggatgcactgggtcaggcaggctcccggccagggcctggagtggatgggcgagatcgaccccagcgacagcaacaccaactacaaccagaagttcaagggcagggtgaccatgaccagggacaccagcatcagcaccgcctacatggaactgagcaggctgaggtccgacgacaccgccgtgtactattgcgccagggaactcctgcacgccgtgtactgggggcagggaacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaagtga

SEQ ID NO: 33 - DNA, 1341 bp, Artificial Sequence
  Chimeric
caggtgcagctggtgcagagcggcgccgaggtgaagaagcccggcgccagcgtgaaagtgagctgcaaggccagcggctacaccttcaccagctactggatgcactgggtcaggcaggctcccggccagggcctggagtggatgggcgagatcgaccccagcgacagcaacaccaactacaaccagaagttcaagggcaaggtgaccatgaccagggacaccagcatcagcaccgcctacatggaactgagcaggctgaggtccgacgacaccgccgtgtactattgcgccagggaactcctgcacgccgtgtactgggggcagggaacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaagtga

SEQ ID NO: 34 - DNA, 642 bp, Artificial Sequence
  Chimeric
gacatccagatgacccagtctcccagcagcctgagcgccagcgtgggcgacagggtgaccattacctgcaccgccagccaggacatccacaagtacatctcctggtaccagcagaagcccggcaaggcccccaagctgctgatctacgacactagcaccctgcagcccggcgtcccttcaaggttcagcggaagcggcagcggcaccgacttcaccttcaccatcagcagcctgcagcccgaggatatcgccacctactactgcctgcagtacgacaacctctggaccttcggccagggcaccaaagtggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgctga

SEQ ID NO: 35 - DNA, 642 bp, Artificial Sequence
  Chimeric
gacatccagatgacccagtctcccagcagcctgagcgccagcgtgggcgacagggtgaccattacctgcaccgccagccaggacatccacaagtacatctcctggtaccagcagaagcccggcaaggcccccaagctgctgatccactacactagcaccctgcagcccggcgtcccttcaaggttcagcggaagcggcagcggcaccgacttcaccttcaccatcagcagcctgcagcccgaggatatcgccacctactactgcctgcagtacgacaacctctggaccttcggccagggcaccaaagtggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgctga

SEQ ID NO: 36 - DNA, 642 bp, Artificial Sequence
  Chimeric
gacatccagatgacccagtctcccagcagcctgagcgccagcgtgggcgacagggtgaccattacctgcaccgccagccaggacatccacaagtacatctcctggtaccagcagaagcccggcaaggcccccaagctgctgatccactacactagcaccctgcagcccggcgtcccttcaaggttcagcggaagcggcagcggcaccgactacaccttcaccatcagcagcctgcagcccgaggatatcgccacctactactgcctgcagtacgacaacctctggaccttcggccagggcaccaaagtggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgctga

SEQ ID NO: 37 - protein, 446 aa, Artificial Sequence
  Chimeric
QVQLVQSGAEVKKPGASVKVSCKASGYTFTSYWMHWVRQAPGQGLEWMGEIDPSDSNTNYNQKFKGRVTMTRDTSISTAYMELSRLRSDDTAVYYCARELLHAVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 446 aa, Artificial Sequence
  Chimeric
QVQLVQSGAEVKKPGASVKVSCKASGYTFTSYWMHWVRQAPGQGLEWMGEIDPSDSNTNYNQKFKGKVTMTRDTSISTAYMELSRLRSDDTAVYYCARELLHAVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 213 aa, Artificial Sequence
  Chimeric
DIQMTQSPSSLSASVGDRVTITCTASQDIHKYISWYQQKPGKAPKLLIYDTSTLQPGVPSRFSGSGSGTDFTFTISSLQPEDIATYYCLQYDNLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 213 aa, Artificial Sequence
  Chimeric
DIQMTQSPSSLSASVGDRVTITCTASQDIHKYISWYQQKPGKAPKLLIHYTSTLQPGVPSRFSGSGSGTDFTFTISSLQPEDIATYYCLQYDNLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 213 aa, Artificial Sequence
  Chimeric
DIQMTQSPSSLSASVGDRVTITCTASQDIHKYISWYQQKPGKAPKLLIHYTSTLQPGVPSRFSGSGSGTDYTFTISSLQPEDIATYYCLQYDNLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - DNA, 321 bp, Artificial Sequence
  Chimeric
gacatccagatgacccagtctcccagcagcctgagcgccagcgtgggcgacagggtgaccattacctgcaccgccagccaggacatccacaagtacatctcctggtaccagcagaagcccggcaaggcccccaagctgctgatctacgacactagcaccctgcagcccggcgtcccttcaaggttcagcggaagcggcagcggcaccgacttcaccttcaccatcagcagcctgcagcccgaggatatcgccacctactactgcctgcagtacgacaacctctggaccttcggccagggcaccaaagtggagatcaagcgt

SEQ ID NO: 43 - protein, 107 aa, Artificial Sequence; identical to SEQ ID NO: 24
  Chimeric

(19) United States
(12) Patent Application Publication
(10) Pub. No.: US 2011/0105724 A1
(43) Pub. Date: May 5, 2011

(54) NOVEL COMPOUNDS

(76) Inventors: Stephanie Jane Clegg, Hertfordshire (GB); Jonathan H. Ellis, Hertfordshire (GB); Paul Andrew Hamblin, Hertfordshire (GB); Karen Fran Kozarsky, King of Prussia, PA (US); Jui-Lan Su, Durham, NC (US); Deborah Ann Welham, King of Prussia, PA (US)

(21) Appl. No.: 12/673,599

(22) PCT Filed: Aug. 15, 2008
(86) PCT No.: PCT/US08/73241

§ 371 Date: Feb. 16, 2010

Related U.S. Application Data
(60) Provisional application No. 60/956,181, filed on Aug. 16, 2007.

(51) Int. Cl. C07K 16/00; C12N 5/04; C12N 15/63; C12P 21/04; C12N 5/07; C12N 1/00

(52) U.S. Cl. 530/387.1; 435/419; 435/320.1; 435/69.6; 435/326; 435/243

(57) ABSTRACT

The present invention relates to neutralizing antibodies (immunoglobulins) which are specific for human IL-8 and bind to as well as neutralize human IL-8.

### CROSS REFERENCE TO RELATED APPLICATION

This application claims priority to U.S. application 60/956,181, filed 16 Aug. 2007, which is incorporated by reference in its entirety.

### FIELD OF THE INVENTION

The present invention relates to neutralizing antibodies which are specific for interleukin-8 (IL-8). In particular the antibodies of the present invention bind to human IL-8. The present invention is also concerned with methods of preventing or treating diseases or disorders characterised by elevated or unbalanced levels of human IL-8, particularly endometriosis, pneumococcal meningitis, COPD, osteoarthritis, rheumatoid arthritis, inflammatory bowel disease, psoriasis, transplant rejection, gout, cancer, cystic fibrosis, adult respiratory distress syndrome, sepsis, or reperfusion injury with said antibodies.

### BACKGROUND OF THE INVENTION

Published data and reports indicate that IL-8, a member of the CXC chemokine family, is elevated in endometriosis, both in serum and in peritoneal fluid. In the peritoneal fluid of patients with endometriosis, IL-8 levels have been shown to be higher, as compared to controls, and levels correlate with severity of disease (Mol. Hum. Repr. 1996; 2:40-45; J Clin Endocrinol Metab. 2000; 85:824-9; Gynecol. Obstet. Invest. 2002; 54:82-87; Hum. Repr. 2003; 18:593-597). In addition to IL-8, IL-6 and TNF alpha are increased in peritoneal fluid of patients with early and active lesions of endometriosis, suggesting that these cytokines may be involved in the neovascularization of the early stages of endometriosis (J Clin Endocrinol Metab. 2000; 85:824-9). Peritoneal TNF alpha levels decrease at later stages of disease, whereas IL-8 and MCP-1 levels continue to increase (Gynecol. Obstet. Invest. 2002; 54:82-87). Peritoneal fluid IL-8 levels in controls were approximately 33 pg/ml, and increased to 200 pg/ml in Stage III patients. Serum levels of IL-8 are also increased in patients with endometriosis (from control levels of 21 pg/ml to levels averaging from 120 to 180 pg/ml in endometriosis patients), but levels do not appear to correlate with the stage of disease (Gynecol. Obstet. Invest. 2002; 54:82-87).

Analysis of endometrium directly isolated from patients showed an increase in IL-8 mRNA and expression (Ann N Y Acad. Sci. 2002; 955:101-9). In addition, expression of the two receptors for IL-8, CXCR1 and CXCR2, is up-regulated in both eutopic and ectopic endometrium of women with endometriosis, as compared to normal endometrium (Human Reproduction 2005; 20:794-801).

It has been postulated that prolonged and elevated expression of IL-8 could be involved in the development of diseases such as endometriosis, COPD, adult respiratory distress syndrome, rheumatoid arthritis, asthma, and other inflammatory diseases as well as in diseases which require neovascularization such as cancer. IL-8 is known to stimulate neutrophil chemotaxis by engaging and activating the CXCR1 and/or CXCR2 receptors, and also to stimulate endometrial endothelial and stromal cell proliferation. Thus the inhibition of IL-8 could prevent inflammatory cells from infiltrating the endometriotic lesions and thus prevent tissue damage, and could prevent proliferation of ectopic endometrial cells as well as angiogenesis of the lesions. The present invention is directed to inhibiting the activation of CXCR1 and CXCR2 receptors by using an antibody having the ability to bind to and neutralize human IL-8.

### SUMMARY OF THE INVENTION

The present invention relates to neutralizing antibodies (immunoglobulins) which are specific for human IL-8 and bind to as well as neutralize human IL-8. The present invention is also concerned with methods of preventing or treating diseases or disorders characterised by elevated or unbalanced levels of human IL-8, particularly endometriosis, COPD, pneumococcal meningitis, osteoarthritis, rheumatoid arthritis, inflammatory bowel disease, psoriasis, gout, cancer, cystic fibrosis, adult respiratory distress syndrome, sepsis, or reperfusion injury with said antibodies.

### DETAILED DESCRIPTION

In one aspect, the present invention relates to an isolated neutralizing antibody which is specific for human IL-8; thus an antibody of the present invention is an anti-IL-8 specific antibody. An anti-IL-8 specific antibody may sometimes be referred to herein as an IL-8 antibody. The definition of the antibody includes the antigen binding portion (or fragment) of the antibody; such antibody portion (or fragment) binds to and neutralizes human IL-8. The antibody of the invention is preferably monoclonal, chimeric, human or humanized.

Preferably, an anti-IL8 specific antibody of the present invention binds to human IL-8 with an equilibrium constant, KD, value of less than 10⁻⁷ M, more preferably less than 10⁻⁸ M, even more preferably less than 10⁻⁹M, or yet even more preferably less than 10⁻¹⁰ M as determined by surface plasmon resonance. Typically surface plasmon resonance measurement is conducted as described in Example 4 as set forth below, and in one embodiment, KD values as described herein are obtained by procedures described in Example 4.

In one embodiment the present method comprises decreasing neutrophil activation by a monoclonal IL-8 antibody of the present invention.

In one embodiment, a monoclonal IL-8 antibody of the present invention binds within epitope of KTYSKPFHPKFI (SEQ ID NO: 31) in human IL-8.

In one embodiment, a monoclonal IL-8 antibody of the present invention is generated by a method comprising immunization with human IL-8.

In one embodiment, a monoclonal IL-8 antibody of the present invention is generated by a method comprising co-immunization with human IL-8 and cynomolgus IL-8.

In one embodiment, a monoclonal IL-8 antibody of the present invention is generated by a method comprising co-immunization with human IL-8, Gro-alpha, Gro-beta, Gro-gamma, and ENA-78, comprising the steps of
a. immunizing mice with a mixture of chemokines IL-8, Gro-alpha, Gro-beta, Gro-gamma, and ENA-78;
b. isolating B cells from the mouse;
c. fusing the B cells with myeloma cells to form immortal hybridoma cells that secrete the desired antibody; and
d. isolating the antibody from the culture supernatant of the hybridoma.

In one embodiment, an antibody of the present invention comprises heavy and light chain variable regions encoded by nucleotide sequences comprising sequences as set forth in SEQ ID NO:1 and SEQ ID NO:2, respectively; or one or more nucleotide sequences can be at least 90%, 95%, 98% or 99% identical to the nucleotide sequences as set forth in SEQ ID NO:1 and SEQ ID NO:2, respectively or conservative sequence modifications thereof.

In one embodiment, an antibody of the present invention comprises heavy and light chain variable regions comprising amino acid sequences as set forth in SEQ ID NO:3 and SEQ ID NO:4, respectively, or one or more conservative sequence modifications thereof.

In one embodiment, an antibody of the present invention comprises heavy and light chain variable regions comprising polypeptides which are at least 90%, 95%, 98% or 99% identical to the amino acid sequences as set forth in SEQ ID NO:3 and SEQ ID NO:4, respectively.

In one embodiment, an antibody of the present invention comprises heavy chain variable region comprising the amino acid sequence as set forth in SEQ ID NO:3 or a conservative sequence modification thereof.

In one embodiment, an antibody of the present invention comprises light chain variable region comprising the amino acid sequence as set forth in SEQ ID NO:4 or a conservative sequence modification thereof.

In one embodiment, an antibody of the present invention comprises heavy chain variable region which is at least 90%, 95%, 98% or 99% identical to the amino acid sequence as set forth in SEQ ID NO: 3.

In one embodiment, an antibody of the present invention comprises light chain variable region which is at least 90%, 95%, 98% or 99% identical to the amino acid sequence as set forth in SEQ ID NO: 4.

In one embodiment, an antibody of the present invention comprises CDR sequences of SEQ ID NOs: 5, 6, 7, 8, 9, and 10; or one or more of the CDR sequences can be conservative sequence modifications of the sequences as set forth in sequences SEQ ID NOs: 5, 6, 7, 8, 9, and 10; or one or more of the CDR sequences are at least 90%, 95%, 98% or 99% identical to the amino acid sequences as set forth in sequences SEQ ID NOs: 5, 6, 7, 8, 9, and 10.

In one embodiment, an antibody of the present invention comprises at least four CDR sequences selected from the group consisting of SEQ ID NOs: 5, 6, 7, 8, 9, and 10; or one or more of the CDR sequences can be conservative sequence modifications of the sequences as set forth in sequences SEQ ID NOs: 5, 6, 7, 8, 9, and 10; or one or more of the CDR sequences are at least 90%, 95%, 98% or 99% identical to the amino acid sequences as set forth in sequences SEQ ID NOs: 5, 6, 7, 8, 9, and 10.

In one embodiment, an antibody of the present invention comprises at least three CDR sequences selected from the group consisting of SEQ ID NOs: 5, 6, 7, 8, 9, and 10; or one or more of the CDR sequences can be conservative sequence modifications of the sequences as set forth in sequences SEQ ID NOs: 5, 6, 7, 8, 9, and 10; or one or more of the CDR sequences are at least 90%, 95%, 98% or 99% identical to the amino acid sequences as set forth in sequences SEQ ID NOs: 5, 6, 7, 8, 9, and 10.

In one embodiment, an antibody of the present invention comprises three CDR sequences of SEQ ID NOs: 5, 6, and 7; or one or more of the CDR sequences can be conservative sequence modifications of the sequences as set forth in sequences SEQ ID NOs: 5, 6, and 7; or one or more of the CDR sequences are at least 90%, 95%, 98% or 99% identical to the amino acid sequences as set forth in sequences SEQ ID NOs: 5, 6, and 7.

In one embodiment, an antibody of the present invention comprises three CDR sequences of SEQ ID NOs: 8, 9, and 10; or one or more of the CDR sequences can be conservative sequence modifications of the sequences as set forth in sequences SEQ ID NOs: 8, 9, and 10; or one or more of the CDR sequences are at least 90%, 95%, 98% or 99% identical to the amino acid sequences as set forth in sequences SEQ ID NOs: 8, 9, and 10.

In one embodiment, an antibody of the present invention comprises at least one CDR sequence selected from the group consisting of (i) SEQ ID NO: 5, 6, 7, 8, 9, and 10; or (ii) one or more conservative sequence modifications of the sequences listed in (i).

In one embodiment, the present invention relates to a hybridoma which produces an antibody comprising CDR sequences of SEQ ID NOs: 5, 6, 7, 8, 9, and 10; or one or more of the CDR sequences can be conservative sequence modifications of the sequences as set forth in sequences SEQ ID NOs: 5, 6, 7, 8, 9, and 10; or one or more of the CDR sequences are at least 90%, 95%, 98% or 99% identical to the amino acid sequences as set forth in sequences SEQ ID NOs: 5, 6, 7, 8, 9, and 10.

In one embodiment, the present invention relates to a host cell (including, but not limited to, a recombinant eukaryotic or prokaryotic cell) which produces an antibody comprising CDR sequences of SEQ ID NOs: 5, 6, 7, 8, 9, and 10; or one or more of the CDR sequences can be conservative sequence modifications of the sequences as set forth in sequences SEQ ID NOs: 5, 6, 7, 8, 9, and 10; or one or more of the CDR sequences are at least 90%, 95%, 98% or 99% identical to the amino acid sequences as set forth in sequences SEQ ID NOs: 5, 6, 7, 8, 9, and 10.

In one embodiment, the present invention relates to a hybridoma which produces an antibody comprising CDR sequences of SEQ ID NOs: 5, 6, and 7; or one or more of the CDR sequences can be conservative sequence modifications of the sequences as set forth in sequences SEQ ID NOs: 5, 6, and 7.

In one embodiment, the present invention relates to a host cell (including, but not limited to, a recombinant eukaryotic or prokaryotic host cell) which produces an antibody comprising CDR sequences of SEQ ID NOs: 5, 6, and 7; or one or more of the CDR sequences can be conservative sequence modifications of the sequences as set forth in sequences SEQ ID NOs: 5, 6, and 7.

In one embodiment, the present invention relates to a hybridoma which produces an antibody comprising CDR sequences of SEQ ID NOs: 8, 9, and 10; or one or more of the CDR sequences can be conservative sequence modifications of the sequences as set forth in sequences SEQ ID NOs: 8, 9, and 10.

In one embodiment, the present invention relates to a host cell (including, but not limited to, a recombinant eukaryotic or prokaryotic host cell) which produces an antibody comprising CDR sequences of SEQ ID NOs: 8, 9, and 10; or one or more of the CDR sequences can be conservative sequence modifications of the sequences as set forth in sequences SEQ ID NOs: 8, 9, and 10.

In one embodiment, the present invention concerns a hybridoma which produces a monoclonal antibody comprising a heavy or light chain variable region encoded by nucleotide sequence comprising a nucleotide sequence as set forth in SEQ ID NO: 1 or SEQ ID NO: 2, respectively.

In one embodiment, the present invention concerns a hybridoma which produces a monoclonal antibody comprising a heavy or light chain variable region encoded by nucleotide sequence comprising nucleotide sequence which is at least 90%, 95%, 98% or 99% identical to a nucleotide sequence as set forth in SEQ ID NO: 1 or SEQ ID NO: 2, respectively.

In one embodiment, the present invention concerns a hybridoma which produces a monoclonal antibody comprising a heavy or light chain variable region comprising the amino acid sequence as set forth in SEQ ID NO:3 or SEQ ID NO: 4, respectively.

In one embodiment, the present invention concerns a hybridoma which produces a monoclonal antibody comprising a heavy or light chain variable region comprising an amino acid sequence which is at least 90%, 95%, 98% or 99% identical to a sequence as set forth in SEQ ID NO:3 or SEQ ID NO: 4, respectively.

In one embodiment, the present invention relates to a host cell (including, but not limited to, a recombinant eukaryotic or prokaryotic host cell) which produces an antibody comprising a heavy or light variable region comprising the amino acid sequence as set forth in SEQ ID NO:3 or SEQ ID NO:4, respectively, or a conservative sequence modification thereof.

In one embodiment, the present invention concerns a host cell (including, but not limited to, a recombinant eukaryotic or prokaryotic host cell) which produces an antibody comprising a heavy or light chain variable region comprising an amino acid sequence which is at least 90%, 95%, 98% or 99% identical to a sequence as set forth in SEQ ID NO:3 or SEQ ID NO: 4, respectively.

In one embodiment, the present invention relates to an expression vector comprising nucleotide sequences encoding a variable heavy or light chain of an antibody comprising the CDR sequences of SEQ ID NOs: 5, 6, and 7; or SEQ ID NOs: 8, 9, and 10, respectively.

In one embodiment, the present invention relates to an expression vector comprising a nucleotide sequence encoding a CDR sequence of an antibody selected from SEQ ID NO: 5, 6, 7, 8, 9, or 10.

In one embodiment, the present invention relates to an expression vector comprising nucleotide sequences encoding at least four CDR sequences of an antibody selected from the group consisting of SEQ ID NOs: 5, 6, 7, 8, 9 and 10.

In one embodiment, the present invention relates to an expression vector comprising polynucleotide sequences of SEQ ID NOs: 11, 12 and 13, or one or more polynucleotide sequences can be at least 90%, 95%, 98% or 99% identical to a sequence as set forth in SEQ ID NOs: 11, 12 and 13.

In one embodiment, the present invention relates to an expression vector comprising polynucleotide sequences of SEQ ID NOs: 14, 15 and 16, or one or more polynucleotide sequences can be at least 90%, 95%, 98% or 99% identical to a sequence as set forth in SEQ ID NOs: 14, 15 and 16.

In one embodiment, the present invention relates to an expression vector comprising a polynucleotide sequence of SEQ ID NOs: 11, 12 or 13, or one or more polynucleotide sequences can be at least 90%, 95%, 98% or 99% identical to a sequence as set forth in SEQ ID NOs: 11, 12 or 13.

In one embodiment, the present invention relates to an expression vector comprising a polynucleotide sequence of SEQ ID NOs: 14, 15 or 16, or one or more polynucleotide sequences can be at least 90%, 95%, 98% or 99% identical to a sequence as set forth in SEQ ID NOs: 14, 15 or 16.

In one embodiment the present invention relates to an expression vector comprising at least four polynucleotide sequences selected from the group consisting of SEQ ID NOs: 11, 12, 13, 14, 15, and 16.

In one embodiment the present invention relates to a process for producing an antibody in a host cell, comprising the steps of:
(i) introducing said host cell with a first DNA sequence encoding at least the variable domain of the antibody heavy chain comprising CDR domains of SEQ ID NOs: 5, 6, and 7; and a second DNA sequence encoding at least the variable domain of the antibody light chain comprising CDR domains of SEQ ID NOs: 8, 9, and 10; and
(ii) expressing said first DNA sequence and said second DNA sequence so that said antibody heavy and light chains are produced in said host cell;

furthermore, this process can be carried out such that said first and second DNA sequences are present in different vectors or said first and second DNA sequences are present in a single vector.

In one embodiment, an antibody of the present invention comprises heavy chain variable region comprising the amino acid sequence as set forth in SEQ ID NO: 22 or 23, or a conservative sequence modification thereof.

In one embodiment, an antibody of the present invention comprises light chain variable region comprising the amino acid sequence as set forth in SEQ ID NO:24, 25 or 26, or a conservative sequence modification thereof.

In one embodiment, an antibody of the present invention comprises heavy chain variable region which is at least 90%, 95%, 98% or 99% identical to the amino acid sequence as set forth in SEQ ID NO: 22 or 24.

In one embodiment, an antibody of the present invention comprises light chain variable region which is at least 90%, 95%, 98% or 99% identical to the amino acid sequence as set forth in SEQ ID NO: 24, 25, or 26.

In one embodiment, the present invention concerns a host cell (including a recombinant eukaryotic or prokaryotic host cell) which produces a monoclonal antibody comprising a heavy or light chain variable region encoded by a nucleotide sequence comprising nucleotide sequence as set forth in SEQ ID NO: 17 or 18, or SEQ ID NO: 19, 20 or 21, respectively.

In one embodiment, the present invention concerns a host cell (including a recombinant eukaryotic or prokaryotic host cell) which produces a monoclonal antibody comprising a heavy or light chain variable region encoded by a nucleotide sequence comprising a nucleotide sequence which is at least 90%, 95%, 98% or 99% identical to a sequence as set forth in SEQ ID NO: 17 or 18, or SEQ ID NO: 19, 20 or 21, respectively.

In one embodiment, the present invention concerns an antibody comprising a heavy or light chain variable region encoded by a nucleotide sequence comprising a nucleotide sequence which is at least 90%, 95%, 98% or 99% identical to a sequence as set forth in SEQ ID NO: 17 or 18, or SEQ ID NO: 19, 20 or 21, respectively.

In one embodiment, the present invention concerns a host cell (including a recombinant eukaryotic or prokaryotic host cell) which produces a monoclonal antibody comprising a heavy or light chain variable region comprising an amino acid sequence as set forth in SEQ ID NO:22 or 23, or SEQ ID NO: 24, 25 or 26, respectively.

In one embodiment, the present invention concerns a host cell (including a recombinant eukaryotic or prokaryotic host cell) which produces a monoclonal antibody comprising a heavy or light chain variable region comprising an amino acid sequence which is at least 90%, 95%, 98% or 99% identical to a sequence as set forth in as set forth in SEQ ID NO:22 or 23, or SEQ ID NO: 24, 25 or 26, respectively.

In one embodiment, the present invention relates to an expression vector comprising a polynucleotide sequence of SEQ ID NO: 17 or 18, or a polynucleotide sequence which is at least 90%, 95%, 98% or 99% identical to a sequence as set forth in as set forth in SEQ ID NO:17 or 18.

In one embodiment, the present invention relates to an expression vector comprising a polynucleotide sequence of SEQ ID NO: 19, 20 or 21, or a polynucleotide sequence which is least 90%, 95%, 98% or 99% identical to a sequence as set forth in as set forth in SEQ ID NO:19, 20 or 21.

In one embodiment, an antibody of the present invention comprises heavy and light chain regions encoded by nucleotide sequences comprising sequences as set forth in SEQ ID NO:29 and SEQ ID NO:30, respectively.

In one embodiment, an antibody of the present invention comprises heavy and light chain regions encoded by nucleotide sequences comprising nucleotide sequences which are at least 90%, 95%, 98% or 99% identical to sequences as set forth in SEQ ID NO:29 and SEQ ID NO:30, respectively.

In one embodiment, an antibody of the present invention comprises heavy and light chain regions comprising amino acid sequences as set forth in SEQ ID NO:27 and SEQ ID NO:28, respectively.

In one embodiment, an antibody of the present invention comprises heavy and light chain regions comprising polypeptides which are at least 90%, 95%, 98% or 99% identical to the amino acid sequences as set forth in SEQ ID NO:27 and SEQ ID NO:28, respectively.

In one embodiment, an antibody of the present invention comprises a heavy chain region comprising the amino acid sequence as set forth in SEQ ID NO:27 or a conservative sequence modification thereof.

In one embodiment, an antibody of the present invention comprises a light chain region comprising the amino acid sequence as set forth in SEQ ID NO:28 or a conservative sequence modification thereof.

In one embodiment, an antibody of the present invention comprises a heavy chain region comprising the amino acid sequence which is at least 90%, 95%, 98% or 99% identical to the amino acid sequence as set forth in SEQ ID NO: 27.

In one embodiment, an antibody of the present invention comprises a light chain region comprising the amino acid sequence which is at least 90%, 95%, 98% or 99% identical to the amino acid sequence as set forth in SEQ ID NO: 28.

In one embodiment the present invention relates to a process for producing an antibody in a host cell, comprising the steps of:
(i) introducing said host cell with a first DNA sequence encoding at least the variable domain of the antibody heavy chain comprising amino acid sequence SEQ ID NO: 22 or 23; and a second DNA sequence encoding at least the variable domain of the antibody light chain comprising amino acid sequence SEQ ID NO: 24, 25 or 26; and
(ii) expressing said first DNA sequence and said second DNA sequence so that said antibody heavy and light chains are produced in said host cell;

furthermore, this process can be carried out such that said first and second DNA sequences are present in different vectors or said first and second DNA sequences are present in a single vector.

In one embodiment, an antibody of the present invention is an antibody which comprises the ability to block the binding of any one of the aforementioned antibodies to an antigen in an ELISA assay.

In one embodiment, an antibody of the present invention comprises heavy and light chains.

In one embodiment, an antibody of the present invention comprises heavy and light chains comprising the amino acid sequences of SEQ ID NO: 37 and SEQ ID NO: 39, respectively.

In one embodiment, an antibody of the present invention comprises heavy and light chains comprising the amino acid sequences of SEQ ID NO: 37 and SEQ ID NO: 40, respectively.

In one embodiment, an antibody of the present invention comprises heavy and light chains comprising the amino acid sequences of SEQ ID NO: 37 and SEQ ID NO: 41, respectively.

In one embodiment, an antibody of the present invention comprises heavy and light chains comprising the amino acid sequences of SEQ ID NO: 38 and SEQ ID NO: 39, respectively.

In one embodiment, an antibody of the present invention comprises heavy and light chains comprising the amino acid sequences of SEQ ID NO: 38 and SEQ ID NO: 40, respectively.

In one embodiment, an antibody of the present invention comprises heavy and light chains comprising the amino acid sequences of SEQ ID NO: 38 and SEQ ID NO: 41, respectively.

In one embodiment, the present invention relates to an antibody that competes with the binding of any one of the aforementioned antibodies to human-IL8.

In one embodiment, the present invention relates to a composition comprising an aforementioned antibody and a pharmaceutically acceptable carrier.

In one embodiment, the present invention relates to a method of treating or preventing in humans endometriosis, COPD, osteoarthritis, pneumococcal meningitis, rheumatoid arthritis, inflammatory bowel disease, psoriasis, gout, cancer, cystic fibrosis, adult respiratory distress syndrome, sepsis, or reperfusion injury comprising administering an effective amount of an aforementioned antibody.

In one embodiment, the present invention relates to an aforementioned antibody for use in preventing and/or treating endometriosis, COPD, osteoarthritis, pneumococcal meningitis, rheumatoid arthritis, inflammatory bowel disease, psoriasis, gout, cancer, cystic fibrosis, adult respiratory distress syndrome, sepsis, or reperfusion injury in humans.

In one embodiment, the present invention relates to use of an aforementioned antibody in the manufacture of a medicament for use in preventing and/or treating endometriosis, COPD, osteoarthritis, pneumococcal meningitis, rheumatoid arthritis, inflammatory bowel disease, psoriasis, gout, cancer, cystic fibrosis, adult respiratory distress syndrome, sepsis, or reperfusion injury in humans.

In one embodiment, the present invention relates to use of an aforementioned antibody in the manufacture of a medicament for preventing and/or treating endometriosis, COPD, osteoarthritis, pneumococcal meningitis, rheumatoid arthritis, inflammatory bowel disease, psoriasis, gout, cancer, cystic fibrosis, adult respiratory distress syndrome, sepsis, or reperfusion injury in humans.

Among the cancers which can be treated by an aforementioned antibody include, but not limited to, glioblastoma, malignant mesothelioma, metastatic melanoma, metastatic breast cancer (estrogen-receptor-negative), metastatic pancreatic cancer, androgen-independent prostate cancer, and ovarian cancer.

### DETAILED DESCRIPTION OF THE INVENTION

An “IL-8 specific antibody” or “anti-IL-8 specific antibody” or “IL-8 antibody,” as used herein, is intended to refer to a neutralizing antibody that binds to human IL-8 and is substantially free of other antibodies having different antigenic specificities, and furthermore, is a single composition of matter. For avoidance of doubt, the IL-8-specific antibody of the present invention need not bind solely to human IL-8, but it may also happen to bind to other non-human orthologues such as cynomolgus, guinea pig or rabbit IL-8. Moreover, an isolated IL-8-specific antibody is substantially free of other cellular material and/or chemicals.

As used herein, “antibody” is also referred to as “immunoglobulin”.

A “neutralizing antibody”, as used is intended to refer to an antibody whose binding to a particular antigen results in inhibition of the biological activity of the antigen. In this instance, one of the biological activities inhibited is full or partial inactivation of neutrophil activation. This inhibition of the biological activity of the antigen can be assessed by measuring one or more indicators of biological activity of the antigen using an appropriate in vitro, ex vivo or in vivo assay as described below (see for example, Example 3.)

One way of measuring the binding kinetics of an antibody is by surface plasmon resonance. The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jonsson, U., et al. (1993) Ann. Biol. Clin. 51:19-26; Jonsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “epitope” means a protein determinant capable of specific binding to an antibody. An epitopes usually consists of chemically active surface grouping of molecules such as amino acids or sugar side chains which usually have specific three dimensional structural characteristics, as well as specific charge characteristics. Conformational and nonconformational epitopes are distinguished in that the binding to the former but not the latter is lost in the presence of denaturing solvents.

A “monoclonal antibody” as opposed to polyclonal antibody refers to antibody composition of single molecular composition. For example, a monoclonal antibody can be derived using a hybridoma-derived antibody method (e.g., such as the hybridoma methodology originally described by Kohler and Milstein (1975, Nature 256:495-497, see also, Brown et al. (1981) J. Immunol. 127:539-46; Brown et al. (1980) J Biol Chem 255:4980-83; Yeh et al. (1976) PNAS 76:2927-31; and Yeh et al. (1982) Int. J. Cancer 29:269-75). The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet., 3:231-36).

A recombinant eukaryotic host cell which can express the antibodies of the present invention include cells such as CHO cells, NS/0 cells, HEK293 cells, PER.C6 cells, plant cells, or fungi, including yeast cells.

As used herein, “specific” binding refers to antibody binding to a predetermined antigen. Typically, the antibody binds with an equilibrium constant, KD, corresponding to about 1×10⁻⁷ M or less, and binds to the predetermined antigen with an affinity corresponding to a KD that is at least two orders of magnitude lower than its affinity for binding to a non-specific antigen (e.g., BSA, casein) other than the predetermined antigen or a closely-related antigen. For avoidance of doubt, “specific” does not preclude the binding of related antigens. In one embodiment, an anti-IL8 specific antibody of the present invention binds not only to human IL-8, but also its non-human orthologues, such as cynomolgus, guinea pig or rabbit IL-8. The phrases “an antibody recognizing an antigen” and “an antibody specific for an antigen” are used interchangeably herein with the term “an antibody which binds specifically to an antigen”.

As used herein, the term “kd” (sec-1), as used herein, is intended to refer to the dissociation rate constant of a particular antibody-antigen interaction.

The term “ka” (M x sec-1), as used herein, is intended to refer to the association rate constant of a particular antibody-antigen interaction.

The term “KD” (M), as used herein, is intended to refer to the equilibrium constant of a particular antibody-antigen interaction and is obtained by dividing the kd by the ka.

“Conservative sequence modifications” for nucleotide and amino acid sequence modifications means changes which do not significantly affect or alter the binding characteristics of the antibody encoded by the nucleotide sequence or containing the amino acid sequence. Such conservative sequence modifications include nucleotide and amino acid substitutions, additions and deletions. Modifications can be introduced into the sequences by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an antibody for which sequence is specifically disclosed is preferably replaced with another amino acid residue from the same side chain family. Thus in one aspect, the IL-8-specific antibody of the present invention includes all the conservative sequence modifications of the specifically disclosed amino acid sequences.

The present invention also encompasses “derivatives” of the amino acid sequences as specifically disclosed, wherein one or more of the amino acid residues have been derivatized, e.g., by acylation or glycosylation, without significantly affecting or altering the binding characteristics of the antibody containing the amino acid sequences.

For nucleic acids, the term “substantial homology” or “substantial identity” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial homology (substantial identity) exists when the segments will hybridize under selective hybridization conditions, to the complement of the strand.

For nucleotide and amino acid sequences, the term “homologous” (or “identical)” indicates the degree of identity between two nucleic acid or amino acid sequences when optimally aligned and compared with appropriate insertions or deletions. Alternatively, substantial homology (identity) exists when the DNA segments will hybridize under selective hybridization conditions, to the complement of the strand.

The percent identity (or percent homology) between two sequences is a function of the number of identical positions shared by the sequences (i.e., % identity=# of identical positions/total # of positions times 100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity (or percent homology) between two nucleotide sequences can be determined using the GAP program in the GCG software package, using a NWSgapdna.CMP matrix and a gap weight of 40, 50, 60, 70, or 80 and a length weight of 1, 2, 3, 4, 5, or 6. The percent identity (percent homology) between two nucleotide or amino acid sequences can also be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

By way of example, a polynucleotide sequence of the present invention may be identical to the reference sequence of SEQ ID NO:2, that is be 100% identical, or it may include up to a certain integer number of nucleotide alterations as compared to the reference sequence. Such alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in SEQ ID NO:2 by the numerical percent of the respective percent identity (divided by 100) and subtracting that product from said total number of nucleotides in SEQ ID NO:2, or:

nn≦xn−(xn·y),

wherein nn is the number of nucleotide alterations, xn is the total number of nucleotides in SEQ ID NO:2, and y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and wherein any non-integer product of xn and y is rounded down to the nearest integer prior to subtracting it from xn. Alterations of the polynucleotide sequence of SEQ ID NO:2 may create nonsense, missense or frameshift mutations in this coding sequence and thereby alter the polypeptide encoded by the polynucleotide following such alterations.

Similarly, in another example, a polypeptide sequence of the present invention may be identical to the reference sequence encoded by SEQ ID NO:3, that is be 100% identical, or it may include up to a certain integer number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the polypeptide sequence encoded by SEQ ID NO:3 by the numerical percent of the respective percent identity (divided by 100) and then subtracting that product from said total number of amino acids in the polypeptide sequence encoded by SEQ ID NO:3, or:

na≦xa−(xa·y),

wherein na is the number of amino acid alterations, xa is the total number of amino acids in the polypeptide sequence encoded by SEQ ID NO:3, and y is, for instance 0.70 for 70%, 0.80 for 80%, 0.85 for 85% etc., and wherein any non-integer product of xa and y is rounded down to the nearest integer prior to subtracting it from xa.

A nucleic acid is “operably linked” when it is placed into a functional relationship with another nucleic acid sequence. For instance, a promoter or enhancer is operably linked to a coding sequence if it affects the transcription of the sequence. With respect to transcription of regulatory sequences, operably linked means that the DNA sequences being linked are contiguous and, where necessary to join two protein coding regions, contiguous and in reading frame. For switch sequences, operably linked indicates that the sequences are capable of effecting switch recombination.

The term “vector,” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. Recombinant host cells include, for example, transfectomas, such as CHO cells, NS/0 cells, and lymphocytic cells.

As used herein, the term “subject” includes any human or non-human animal. The term “non-human animal” includes all vertebrates, e.g., mammals and non-mammals, such as non-human primates, sheep, dog, cow, chickens, amphibians, reptiles, etc.

### 1. Antibody Structures

### Intact Antibodies

Intact antibodies are usually heteromultimeric glycoproteins comprising at least two heavy and two light chains. Aside from IgM, intact antibodies are heterotetrameric glycoproteins of approximately 150 Kda, composed of two identical light (L) chains and two identical heavy (H) chains. Typically, each light chain is linked to a heavy chain by one covalent disulfide bond while the number of disulfide linkages between the heavy chains of different immunoglobulin isotypes varies. Each heavy and light chain also has intrachain disulfide bridges. Each heavy chain has at one end a variable domain (V_{H}) followed by a number of constant regions. Each light chain has a variable domain (V_{L}) and a constant region at its other end; the constant region of the light chain is aligned with the first constant region of the heavy chain and the light chain variable domain is aligned with the variable domain of the heavy chain. The light chains of antibodies from most vertebrate species can be assigned to one of two types called Kappa and Lambda based on the amino acid sequence of the constant region. Depending on the amino acid sequence of the constant region of their heavy chains, human antibodies can be assigned to five different classes, IgA, IgD, IgE, IgG and IgM. IgG and IgA can be further subdivided into subclasses, IgG1, IgG2, IgG3 and IgG4; and IgA1 and IgA2. Species variants exist with mouse and rat having at least IgG2a, IgG2b. The variable domain of the antibody confers binding specificity upon the antibody with certain regions displaying particular variability called complementarity determining regions (CDRs). The more conserved portions of the variable region are called framework regions (FR). The variable domains of intact heavy and light chains each comprise four FR connected by three CDRs. The CDRs in each chain are held together in close proximity by the FR regions and with the CDRs from the other chain contribute to the formation of the antigen binding site of antibodies. The constant regions are not directly involved in the binding of the antibody to the antigen but exhibit various effector functions such as participation in antibody dependent cell-mediated cytotoxicity (ADCC), phagocytosis via binding to Fcγ receptor, half-life/clearance rate via neonatal Fc receptor (FcRn) and complement dependent cytotoxicity via the C1q component of the complement cascade. The human IgG2 constant region lacks the ability to activate complement by the classical pathway or to mediate antibody-dependent cellular cytotoxicity. The IgG4 constant region lacks the ability to activate complement by the classical pathway and mediates antibody-dependent cellular cytotoxicity only weakly. Antibodies essentially lacking these effector functions may be termed ‘non-lytic’ antibodies.

### Human Antibodies

Human antibodies may be produced by a number of methods known to those of skill in the art. Human antibodies can be made by the hybridoma method using human myeloma or mouse-human heteromyeloma cells lines see Kozbor J. Immunol 133, 3001, (1984) and Brodeur, Monoclonal Antibody Production Techniques and Applications, pp 51-63 (Marcel Dekker Inc, 1987). Alternative methods include the use of phage libraries or transgenic mice both of which utilize human V region repertories (see Winter G, (1994), Annu. Rev. Immunol 12, 433-455, Green L L (1999), J. Immunol. methods 231, 11-23).

Several strains of transgenic mice are now available wherein their mouse immunoglobulin loci have been replaced with human immunoglobulin gene segments (see Tomizuka K, (2000) PNAS 97, 722-727; Fishwild D. M (1996) Nature Biotechnol. 14, 845-851, Mendez M J, 1997, Nature Genetics, 15, 146-156). Upon antigen challenge such mice are capable of producing a repertoire of human antibodies from which antibodies of interest can be selected.

Of particular note is the Trimera™ system (see Eren R et al, (1998) Immunology 93:154-161) where human lymphocytes are transplanted into irradiated mice, the Selected Lymphocyte Antibody System (SLAM, see Babcook et al, PNAS (1996) 93:7843-7848) where human (or other species) lymphocytes are effectively put through a massive pooled in vitro antibody generation procedure followed by deconvulated, limiting dilution and selection procedure and the Xenomouse II™ (Abgenix Inc). An alternative approach is available from Morphotek Inc using the Morphodoma™ technology.

Phage display technology can be used to produce human antibodies (and fragments thereof), see McCafferty; Nature, 348, 552-553 (1990) and Griffiths A D et al (1994) EMBO 13:3245-3260. According to this technique antibody V domain genes are cloned in frame into either a major or minor coat of protein gene of a filamentous bacteriophage such as M13 or fd and displayed (usually with the aid of a helper phage) as functional antibody fragments on the surface of the phage particle. Selections based on the functional properties of the antibody result in selection of the gene encoding the antibody exhibiting those properties. The phage display technique can be used to select antigen specific antibodies from libraries made from human B cells taken from individuals afflicted with a disease or disorder described above or alternatively from unimmunized human donors (see Marks; J. Mol. Bio. 222,581-597, 1991). Where an intact human antibody is desired comprising a Fc domain it is necessary to reclone the phage displayed derived fragment into a mammalian expression vectors comprising the desired constant regions and establishing stable expressing cell lines.

The technique of affinity maturation (Marks; Bio/technol 10,779-783 (1992)) may be used to improve binding affinity wherein the affinity of the primary human antibody is improved by sequentially replacing the H and L chain V regions with naturally occurring variants and selecting on the basis of improved binding affinities. Variants of this technique such as “epitope imprinting” are now also available see WO 93/06213. See also Waterhouse; Nucl. Acids Res 21, 2265-2266 (1993).

### Chimaeric and Humanised Antibodies

The use of intact non-human antibodies in the treatment of human diseases or disorders carries with it the now well established problems of potential immunogenicity, especially upon repeated administration of the antibody; that is, the immune system of the patient may recognise the non-human intact antibody as non-self and mount a neutralising response. In addition to developing fully human antibodies (see above) various techniques have been developed over the years to overcome these problems and generally involve reducing the composition of non-human amino acid sequences in the intact therapeutic antibody whilst retaining the relative ease in obtaining non-human antibodies from an immunised animal e.g. mouse, rat or rabbit. Broadly two approaches have been used to achieve this. The first are chimaeric antibodies, which generally comprise a non-human (e.g. rodent such as mouse) variable domain fused to a human constant region. Because the antigen-binding site of an antibody is localised within the variable regions the chimaeric antibody retains its binding affinity for the antigen but acquires the effector functions of the human constant region and are therefore able to perform effector functions such as described supra. Chimaeric antibodies are typically produced using recombinant DNA methods. DNA encoding the antibodies (e.g. cDNA) is isolated and sequenced using conventional procedures (e.g. by using oligonucleotide probes that are capable of binding specifically to genes encoding the H and L chain variable regions of the antibody of the invention. Hybridoma cells serve as a typical source of such DNA. Once isolated, the DNA is placed into expression vectors which are then transfected into host cells such as E. Coli, COS cells, CHO cells or myeloma cells that do not otherwise produce immunoglobulin protein to obtain synthesis of the antibody. The DNA may be modified by substituting the coding sequence for human L and H chains for the corresponding non-human (e.g. murine) H and L constant regions see e.g. Morrison; PNAS 81, 6851 (1984). Thus another embodiment of the invention there is provided a chimaeric antibody comprising a V_{H} domain comprising the sequence: SEQ ID No:3 and a V_{L} domain comprising the sequence: SEQ ID No: 4 fused to a human constant region (which maybe of a IgG isotype e.g. IgG1).

The second approach involves the generation of humanised antibodies wherein the non-human content of the antibody is reduced by humanizing the variable regions. Two techniques for humanisation have gained popularity. The first is humanisation by CDR grafting. CDRs build loops close to the antibody's N-terminus where they form a surface mounted in a scaffold provided by the framework regions. Antigen-binding specificity of the antibody is mainly defined by the topography and by the chemical characteristics of its CDR surface. These features are in turn determined by the conformation of the individual CDRs, by the relative disposition of the CDRs, and by the nature and disposition of the side chains of the residues comprising the CDRs. A large decrease in immunogenicity can be achieved by grafting only the CDRs of non-human (e.g. murine) antibodies (“donor” antibodies) onto a suitable human framework (“acceptor framework”) and constant regions (see Jones et al (1986) Nature 321, 522-525 and Verhoeyen Met al (1988) Science 239, 1534-1536). However, CDR grafting per se may not result in the complete retention of antigen-binding properties and it is frequently found that some framework residues of the donor antibody need to be preserved (sometimes referred to as “backmutations”) in the humanised molecule if significant antigen-binding affinity is to be recovered (see Queen C et al (1989) PNAS 86, 10,029-10,033, Co, Met al (1991) Nature 351, 501-502). In this case, human V regions showing the greatest sequence homology (typically 60% or greater) to the non-human donor antibody may be chosen from a database in order to provide the human framework (FR). The selection of human FRs can be made either from human consensus or individual human antibodies. Where necessary, key residues from the donor antibody are substituted into the human acceptor framework to preserve CDR conformations. Computer modelling of the antibody maybe used to help identify such structurally important residues, see WO99/48523.

Alternatively, humanisation maybe achieved by a process of “veneering”. A statistical analysis of unique human and murine immunoglobulin heavy and light chain variable regions revealed that the precise patterns of exposed residues are different in human and murine antibodies, and most individual surface positions have a strong preference for a small number of different residues (see Padlan E. A. et al; (1991) Mol. Immunol. 28, 489-498 and Pedersen J. T. et al (1994) J. Mol. Biol. 235; 959-973). Therefore it is possible to reduce the immunogenicity of a non-human Fv by replacing exposed residues in its framework regions that differ from those usually found in human antibodies. Because protein antigenicity can be correlated with surface accessibility, replacement of the surface residues may be sufficient to render the mouse variable region “invisible” to the human immune system (see also Mark G. E. et al (1994) in Handbook of Experimental Pharmacology vol. 113: The pharmacology of monoclonal Antibodies, Springer-Verlag, pp 105-134). This procedure of humanisation is referred to as “veneering” because only the surface of the antibody is altered, the supporting residues remain undisturbed. A further alternative approach is set out in WO04/006955.

Further alternative approaches include that set out in WO04/006955 and the procedure of Humaneering™ (Kalobios) which makes use of bacterial expression systems and produces antibodies that are close to human germline in sequence (Alfenito-M Advancing Protein Therapeutics January 2007, San Diego, Calif.). Another, approach to humanisation involves selecting human acceptor frameworks on the basis of structural similarity of the human CDR regions to those of the donor mouse antibody CDR regions rather than on homology between other regions of the antibody such as framework regions. This process is also known as Superhumanisation™ (Evogenix Inc.; Hwang et al (2005) Methods 36:35-42).

It will be apparent to those skilled in the art that the term “derived” is intended to define not only the source in the sense of it being the physical origin for the material but also to define material which is structurally identical to the material but which does not originate from the reference source. Thus “residues found in the donor antibody” need not necessarily have been purified from the donor antibody.

### Bispecific Antibodies

A bispecific antibody is an antibody derivative having binding specificities for at least two different epitopes and also forms part of the invention. Methods of making such antibodies are known in the art. Traditionally, the recombinant production of bispecific antibodies is based on the coexpression of two immunoglobulin H chain-L chain pairs, where the two H chains have different binding specificities see Millstein et al, Nature 305 537-539 (1983), WO93/08829 and Traunecker et al EMBO, 10, 1991, 3655-3659. Because of the random assortment of H and L chains, a potential mixture of ten different antibody structures are produced of which only one has the desired binding specificity. An alternative approach involves fusing the variable domains with the desired binding specificities to heavy chain constant region comprising at least part of the hinge region, CH2 and CH3 regions. It is preferred to have the CH1 region containing the site necessary for light chain binding present in at least one of the fusions. DNA encoding these fusions, and if desired the L chain are inserted into separate expression vectors and are then cotransfected into a suitable host organism. It is possible though to insert the coding sequences for two or all three chains into one expression vector. In one preferred approach, the bispecific antibody is composed of a H chain with a first binding specificity in one arm and a H-L chain pair, providing a second binding specificity in the other arm, see WO94/04690. See also Suresh et al Methods in Enzymology 121, 210, 1986.

### Antibody Fragments

In certain embodiments of the invention there is provided therapeutic antibody which is an antigen binding fragment. Such fragments may be functional antigen binding fragments of intact and/or humanised and/or chimaeric antibodies such as Fab, Fd, Fab′, F(ab′)₂, Fv, ScFv fragments of the antibodies described supra. Fragments lacking the constant region lack the ability to activate complement by the classical pathway or to mediate antibody-dependent cellular cytotoxicity. Traditionally such fragments are produced by the proteolytic digestion of intact antibodies by e.g. papain digestion (see for example, WO 94/29348) but may be produced directly from recombinantly transformed host cells. For the production of ScFv, see Bird et al; (1988) Science, 242, 423-426. In addition, antibody fragments may be produced using a variety of engineering techniques as described below.

Fv fragments appear to have lower interaction energy of their two chains than Fab fragments. To stabilise the association of the V_{H} and V_{L} domains, they have been linked with peptides (Bird et al, (1988) Science 242, 423-426, Huston et al, PNAS, 85, 5879-5883), disulphide bridges (Glockshuber et al, (1990) Biochemistry, 29, 1362-1367) and “knob in hole” mutations (Zhu et al (1997), Protein Sci., 6, 781-788). ScFv fragments can be produced by methods well known to those skilled in the art see Whitlow et al (1991) Methods companion Methods Enzymol, 2, 97-105 and Huston et al (1993) Int. Rev. Immunol 10, 195-217. ScFv may be produced in bacterial cells such as E. Coli but are more typically produced in eukaryotic cells. One disadvantage of ScFv is the monovalency of the product, which precludes an increased avidity due to polyvalent binding, and their short half-life. Attempts to overcome these problems include bivalent (ScFv′)₂ produced from ScFV containing an additional C terminal cysteine by chemical coupling (Adams et al (1993) Can. Res 53, 4026-4034 and McCartney et al (1995) Protein Eng. 8, 301-314) or by spontaneous site-specific dimerization of ScFv containing an unpaired C terminal cysteine residue (see Kipriyanov et al (1995) Cell. Biophys 26, 187-204). Alternatively, ScFv can be forced to form multimers by shortening the peptide linker to between 3 to 12 residues to form “diabodies”, see Holliger et al PNAS (1993), 90, 6444-6448. Reducing the linker still further can result in ScFV trimers (“triabodies”, see Kortt et al (1997) Protein Eng, 10, 423-433) and tetramers (“tetrabodies”, see Le Gall et al (1999) FEBS Lett, 453, 164-168). Construction of bivalent ScFV molecules can also be achieved by genetic fusion with protein dimerizing motifs to form “miniantibodies” (see Pack et al (1992) Biochemistry 31, 1579-1584) and “minibodies” (see Hu et al (1996), Cancer Res. 56, 3055-3061). ScFv-Sc-Fv tandems ((ScFV)₂) may also be produced by linking two ScFv units by a third peptide linker, see Kurucz et al (1995) J. Immol. 154, 4576-4582. Bispecific diabodies can be produced through the noncovalent association of two single chain fusion products consisting of V_{H} domain from one antibody connected by a short linker to the V_{L} domain of another antibody, see Kipriyanov et al (1998), Int. J. Can 77, 763-772. The stability of such bispecific diabodies can be enhanced by the introduction of disulphide bridges or “knob in hole” mutations as described supra or by the formation of single chain diabodies (ScDb) wherein two hybrid ScFv fragments are connected through a peptide linker see Kontermann et al (1999) J. Immunol. Methods 226 179-188. Tetravalent bispecific molecules are available by e.g. fusing a ScFv fragment to the CH3 domain of an IgG molecule or to a Fab fragment through the hinge region see Coloma et al (1997) Nature Biotechnol. 15, 159-163. Alternatively, tetravalent bispecific molecules have been created by the fusion of bispecific single chain diabodies (see Alt et al, (1999) FEBS Lett 454, 90-94. Smaller tetravalent bispecific molecules can also be formed by the dimerization of either ScFv-ScFv tandems with a linker containing a helix-loop-helix motif (DiBi miniantibodies, see Muller et al (1998) FEBS Lett 432, 45-49) or a single chain molecule comprising four antibody variable domains (V_{H} and V_{L}) in an orientation preventing intramolecular pairing (tandem diabody, see Kipriyanov et al, (1999) J. Mol. Biol. 293, 41-56). Bispecific F(ab′)2 fragments can be created by chemical coupling of Fab′ fragments or by heterodimerization through leucine zippers (see Shalaby et al, (1992) J. Exp. Med. 175, 217-225 and Kostelny et al (1992), J. Immunol. 148, 1547-1553). Also available are so-called domain antibodies based on isolated V_{H} or V_{L} domains (Domantis Ltd.), see U.S. Pat. No. 6,248,516; U.S. Pat. No. 6,291,158; U.S. Pat. No. 6,172,197.

### Heteroconjugate Antibodies

Heteroconjugate antibodies are derivatives which also form an embodiment of the present invention. Heteroconjugate antibodies are composed of two covalently joined antibodies formed using any convenient cross-linking methods. See U.S. Pat. No. 4,676,980.

### Other Modifications

Antibodies of the present invention may also incorporate any other modifications in the constant regions. For example glycosylation of antibodies at conserved positions in their constant regions is known to have a profound effect on antibody function, particularly effector functioning such as those described above, see for example, Boyd et al (1996), Mol. Immunol. 32, 1311-1318. Glycosylation variants of the therapeutic antibodies of the present invention wherein one or more carbohydrate moiety is added, substituted, deleted or modified are contemplated. Introduction of an asparagine-X-serine or asparagine-X-threonine motif creates a potential site for enzymatic attachment of carbonhydrate moieties and may therefore be used to manipulate the glycosylation of an antibody. In Raju et al (2001) Biochemistry 40, 8868-8876 the terminal sialyation of a TNFR-IgG immunoadhesin was increased through a process of regalactosylation and/or resialylation using beta-1,4-galactosyltransferace and/or alpha, 2,3 sialyltransferase. Increasing the terminal sialylation is believed to increase the half-life of the immunoglobulin. Antibodies, in common with most glycoproteins, are typically produced in nature as a mixture of glycoforms. This mixture is particularly apparent when antibodies are produced in eukaryotic, particularly mammalian cells. A variety of methods have been developed to manufacture defined glycoforms, see Zhang et al Science (2004), 303, 371, Sears et al, Science, (2001) 291, 2344, Wacker et al (2002) Science, 298 1790, Davis et al (2002) Chem. Rev. 102, 579, Hang et al (2001) Acc. Chem. Res 34, 727. Thus the invention concerns a plurality of therapeutic antibodies (which maybe of the IgG isotype, e.g. IgG1) as described herein comprising a defined number (e.g. 7 or less, for example 5 or less such as two or a single) glycoform(s) of said antibodies.

Derivatives according to the invention also include therapeutic antibodies of the invention coupled to a non-proteinaeous polymer such as polyethylene glycol (PEG), polypropylene glycol or polyoxyalkylene. Conjugation of proteins to PEG is an established technique for increasing half-life of proteins, as well as reducing antigenicity and immunogenicity of proteins. The use of PEGylation with different molecular weights and styles (linear or branched) has been investigated with intact antibodies as well as Fab′ fragments, see Koumenis I. L. et al (2000) Int. J. Pharmaceut. 198:83-95. A particular embodiment comprises an antigen-binding fragment of the invention without the effector functions of a) activation of complement by the classical pathway; and b) mediating antibody-dependent cellular cytotoxicity; (such as a Fab fragment or a scFv) coupled to PEG.

### 2. Production Methods

Antibodies of the present invention may be produced in transgenic organisms such as goats (see Pollock et al (1999), J. Immunol. Methods 231:147-157), chickens (see Morrow K J J (2000) Genet. Eng. News 20:1-55), mice (see Pollock et al ibid) or plants (see Doran P M, (2000) Curr. Opinion Biotechnol. 11, 199-204, Ma JK-C (1998), Nat. Med. 4; 601-606, Baez J et al, BioPharm (2000) 13: 50-54, Stoger E et al; (2000) Plant Mol. Biol. 42:583-590). Antibodies may also be produced by chemical synthesis. However, antibodies of the invention are typically produced using recombinant cell culturing technology well known to those skilled in the art. A polynucleotide encoding the antibody is isolated and inserted into a replicable vector such as a plasmid for further cloning (amplification) or expression in a host cell. One useful expression system is a glutamate synthetase system (such as sold by Lonza Biologics), particularly where the host cell is CHO or NS0 (see below). Polynucleotide encoding the antibody is readily isolated and sequenced using conventional procedures (e.g. oligonucleotide probes). Vectors that may be used include plasmid, virus, phage, transposons, minichromsomes of which plasmids are a typical embodiment. Generally such vectors further include a signal sequence, origin of replication, one or more marker genes, an enhancer element, a promoter and transcription termination sequences operably linked to the light and/or heavy chain polynucleotide so as to facilitate expression. Polynucleotide encoding the light and heavy chains may be inserted into separate vectors and introduced (e.g. by transformation, transfection, electroporation or transduction) into the same host cell concurrently or sequentially or, if desired both the heavy chain and light chain can be inserted into the same vector prior to such introduction.

It will be immediately apparent to those skilled in the art that due to the redundancy of the genetic code, alternative polynucleotides to those disclosed herein are also available that will encode the polypeptides of the invention.

### Signal Sequences

Antibodies of the present invention may be produced as a fusion protein with a heterologous signal sequence having a specific cleavage site at the N terminus of the mature protein. The signal sequence should be recognised and processed by the host cell. For prokaryotic host cells, the signal sequence may be an alkaline phosphatase, penicillinase, or heat stable enterotoxin II leaders. For yeast secretion the signal sequences may be a yeast invertase leader, α factor leader or acid phosphatase leaders see e.g. WO90/13646. In mammalian cell systems, viral secretory leaders such as herpes simplex gD signal and a native immunoglobulin signal sequence (such as human Ig heavy chain) are available, among others. Typically the signal sequence is ligated in reading frame to polynucleotide encoding the antibody of the invention.

### Origin of Replication

Origin of replications are well known in the art with pBR322 suitable for most gram-negative bacteria, 2μ, plasmid for most yeast and various viral origins such as SV40, polyoma, adenovirus, VSV or BPV for most mammalian cells. Generally the origin of replication component is not needed for integrated mammalian expression vectors, unless vector propagation is required in E. coli. However the SV40 on may be used since it contains the early promoter.

### Selection Marker

Typical selection genes encode proteins that (a) confer resistance to antibiotics or other toxins e.g. ampicillin, neomycin, methotrexate or tetracycline or (b) complement auxotrophic deficiencies or supply nutrients not available in the complex media or (c) combinations of both. The selection scheme may involve arresting growth of the host cells that contain no vector or vectors. Cells, which have been successfully transformed with the genes encoding the therapeutic antibody of the present invention, survive due to e.g. drug resistance conferred by the co-delivered selection marker. One example is the DHFR-selection system wherein transformants are generated in DHFR negative host strains (e.g., see Page and Sydenham 1991 Biotechnology 9: 64-68). In this system the DHFR gene is co-delivered with antibody polynucleotide sequences of the invention and DHFR positive cells then selected by nucleoside withdrawal. If required, the DHFR inhibitor methotrexate is also employed to select for transformants with DHFR gene amplification. By operably linking DHFR gene to the antibody coding sequences of the invention or functional derivatives thereof, DHFR gene amplification results in concomitant amplification of the desired antibody sequences of interest. CHO cells are a particularly useful cell line for this DHFR/methotrexate selection and methods of amplifying and selecting host cells using the DHFR system are well established in the art see Kaufman R. J. et al J. Mol. Biol. (1982) 159, 601-621, for review, see Werner R G, Noe W, Kopp K, Schluter M, “Appropriate mammalian expression systems for biopharmaceuticals”, Arzneimittel-Forschung. 48(8):870-80, 1998 Aug. A further example is the glutamate synthetase expression system (Lonza Biologics). A suitable selection gene for use in yeast is the trp1 gene; see Stinchcomb et al Nature 282, 38, 1979.

### Promoters

Suitable promoters for expressing antibodies of the invention are operably linked to DNA/polynucleotide encoding the antibody. Promoters for prokaryotic hosts include phoA promoter, Beta-lactamase and lactose promoter systems, alkaline phosphatase, tryptophan and hybrid promoters such as Tac. Promoters suitable for expression in yeast cells include 3-phosphoglycerate kinase or other glycolytic enzymes e.g. enolase, glyceralderhyde 3 phosphate dehydrogenase, hexokinase, pyruvate decarboxylase, phosphofructokinase, glucose 6 phosphate isomerase, 3-phosphoglycerate mutase and glucokinase, among others. Inducible yeast promoters include alcohol dehydrogenase 2, isocytochrome C, acid phosphatase, metallothionein and enzymes responsible for nitrogen metabolism or maltose/galactose utilization, among others.

Promoters for expression in mammalian cell systems include RNA polymerase II promoters including viral promoters such as polyoma, fowlpox and adenoviruses (e.g. adenovirus 2), bovine papilloma virus, avian sarcoma virus, cytomegalovirus (in particular the immediate early gene promoter), retrovirus, hepatitis B virus, actin, rous sarcoma virus (RSV) promoter and the early or late Simian virus 40 and non-viral promoters such as EF-1alpha (Mizushima and Nagata Nucleic Acids Res 1990 18(17): 5322, among others. The choice of promoter may be based upon suitable compatibility with the host cell used for expression.

### Enhancer Element

Where appropriate, e.g. for expression in higher eukaroytics, additional enhancer elements can included instead of or as well as those found located in the promoters described above. Suitable mammalian enhancer sequences include enhancer elements from globin, elastase, albumin, fetoprotein, metallothionine and insulin. Alternatively, one may use an enhancer element from a eukaroytic cell virus such as SV40 enhancer, cytomegalovirus early promoter enhancer, polyoma enhancer, baculoviral enhancer or murine IgG2a locus (see WO04/009823). Whilst such enhancers are typically located on the vector at a site upstream to the promoter, they can also be located elsewhere e.g. within the untranslated region or downstream of the polydenalytion signal. The choice and positioning of enhancer may be based upon suitable compatibility with the host cell used for expression.

### Polyadenylation/Termination

In eukaryotic systems, polyadenylation signals are operably linked to polynucleotide encoding the antibody of this invention. Such signals are typically placed 3′ of the open reading frame. In mammalian systems, non-limiting example signals include those derived from growth hormones, elongation factor-1 alpha and viral (eg SV40) genes or retroviral long terminal repeats. In yeast systems non-limiting examples of polydenylation/termination signals include those derived from the phosphoglycerate kinase (PGK) and the alcohol dehydrogenase 1 (ADH) genes. In prokaryotic system polyadenylation signals are typically not required and it is instead usual to employ shorter and more defined terminator sequences. The choice of polyadenylation/termination sequences may be based upon suitable compatibility with the host cell used for expression.

### Other Methods/Elements for Enhanced Yields

In addition to the above, other features that can be employed to enhance yields include chromatin remodelling elements, introns and host-cell specific codon modification. The codon usage of the antibody of this invention thereof can be modified to accommodate codon bias of the host cell such to augment transcript and/or product yield (eg Hoekema A et al Mol Cell Biol 1987 7(8):2914-24). The choice of codons may be based upon suitable compatibility with the host cell used for expression.

### Host Cells

Suitable host cells for cloning or expressing vectors encoding antibodies of the invention are, for example, prokaroytic, yeast or higher eukaryotic cells. Suitable prokaryotic cells include eubacteria e.g. enterobacteriaceae such as Escherichia e.g. E. Coli (for example ATCC 31,446; 31,537; 27,325), Enterobacter, Erwinia, Klebsiella Proteus, Salmonella e.g. Salmonella typhimurium, Serratia e.g. Serratia marcescans and Shigella as well as Bacilli such as B. subtilis and B. lichenifonnis (see DD 266 710), Pseudomonas such as P. aeruginosa and Streptomyces. Of the yeast host cells, Saccharomyces cerevisiae, schizosaccharomyces pombe, Kluyveromyces (e.g. ATCC 16,045; 12,424; 24178; 56,500), yarrowia (EP402, 226), Pichia Pastoris (EP183, 070, see also Peng et al J. Biotechnol. 108 (2004) 185-192), Candida, Trichoderma reesia (EP244, 234), Penicillin, Tolypocladium and Aspergillus hosts such as A. nidulans and A. niger are also contemplated, among others.

Although Prokaryotic and yeast host cells are specifically contemplated by the invention, typically however, host cells of the present invention are vertebrate cells. Suitable vertebrate host cells include mammalian cells such as COS-1 (ATCC No. CRL 1650) COS-7 (ATCC CRL 1651), human embryonic kidney line 293, PerC6 (Crucell), baby hamster kidney cells (BHK) (ATCC CRL. 1632), BHK570 (ATCC NO: CRL 10314), 293 (ATCC NO. CRL 1573), Chinese hamster ovary cells CHO (e.g. CHO-K1, ATCC NO: CCL 61, DHFR-CHO cell line such as DG44 (see Urlaub et al, (1986) ibid), particularly those CHO cell lines adapted for suspension culture, mouse sertoli cells, monkey kidney cells, African green monkey kidney cells (ATCC CRL-1587), HELA cells, canine kidney cells (ATCC CCL 34), human lung cells (ATCC CCL 75), Hep G2 and myeloma or lymphoma cells e.g. NS0 (see for example U.S. Pat. No. 5,807,715), Sp2/0, Y0.

Thus in one embodiment of the invention there is provided a stably transformed host cell comprising a vector encoding a heavy chain and/or light chain of the therapeutic antibody as described herein. Typically such host cells comprise a first vector encoding the light chain and a second vector encoding said heavy chain.

Such host cells may also be further engineered or adapted to modify quality, function and/or yield of the antibody of this invention. Non-limiting examples include expression of specific modifying (eg glycosylation) enzymes and protein folding chaperones.

### Cell Culturing Methods.

Host cells transformed with vectors encoding the therapeutic antibodies of the invention may be cultured by any method known to those skilled in the art. Host cells may be cultured in spinner flasks, shake flasks, roller bottles or hollow fibre systems but it is preferred for large scale production that stirred tank reactors or bag reactors (eg Wave Biotech, Somerset, N.J. USA) are used particularly for suspension cultures. Typically the stirred tankers are adapted for aeration using e.g. spargers, baffles or low shear impellers. For bubble columns and airlift reactors direct aeration with air or oxygen bubbles maybe used. Where the host cells are cultured in a serum free culture media it is preferred that the media is supplemented with a cell protective agent such as pluronic F-68 to help prevent cell damage as a result of the aeration process. Depending on the host cell characteristics, either microcarriers maybe used as growth substrates for anchorage dependent cell lines or the cells maybe adapted to suspension culture (which is typical). The culturing of host cells, particularly vertebrate host cells may utilise a variety of operational modes such as batch, fed-batch, repeated batch processing (see Drapeau et al (1994) cytotechnology 15: 103-109), extended batch process or perfusion culture. Although recombinantly transformed mammalia host cells may be cultured in serum-containing media such media comprising fetal calf serum (FCS), it is preferred that such host cells are cultured in synthetic serum-free media such as disclosed in Keen et al (1995) Cytotechnology 17:153-163, or commercially available media such as ProCHO-CDM or UltraCHO™ (Cambrex N.J., USA), supplemented where necessary with an energy source such as glucose and synthetic growth factors such as recombinant insulin. The serum-free culturing of host cells may require that those cells are adapted to grow in serum free conditions. One adaptation approach is to culture such host cells in serum containing media and repeatedly exchange 80% of the culture medium for the serum-free media so that the host cells learn to adapt in serum free conditions (see e.g. Scharfenberg K et al (1995) in Animal Cell technology: Developments towards the 21st century (Beuvery E. C. et al eds), pp 619-623, Kluwer Academic publishers).

Antibodies of the invention secreted into the media may be recovered and purified from the media using a variety of techniques to provide a degree of purification suitable for the intended use. For example the use of therapeutic antibodies of the invention for the treatment of human patients typically mandates at least 95% purity as determined by reducing SDS-PAGE, more typically 98% or 99% purity, when compared to the culture media comprising the therapeutic antibodies. In the first instance, cell debris from the culture media is typically removed using centrifugation followed by a clarification step of the supernatant using e.g. microfiltration, ultrafiltration and/or depth filtration. Alternatively, the antibody can be harvested by microfiltration, ultrafiltration or depth filtration without prior centrifugation. A variety of other techniques such as dialysis and gel electrophoresis and chromatographic techniques such as hydroxyapatite (HA), affinity chromatography (optionally involving an affinity tagging system such as polyhistidine) and/or hydrophobic interaction chromatography (HIC, see U.S. Pat. No. 5,429,746) are available. In one embodiment, the antibodies of the invention, following various clarification steps, are captured using Protein A or G affinity chromatography followed by further chromatography steps such as ion exchange and/or HA chromatography, anion or cation exchange, size exclusion chromatography and ammonium sulphate precipitation. Typically, various virus removal steps are also employed (e.g. nanofiltration using e.g. a DV-20 filter). Following these various steps, a purified (typically monoclonal) preparation comprising at least 10 mg/ml or greater e.g. 100 mg/ml or greater of the antibody of the invention is provided and therefore forms an embodiment of the invention. Concentration to 100 mg/ml or greater can be generated by ultracentrifugation. Suitably such preparations are substantially free of aggregated forms of antibodies of the invention.

Bacterial systems are particularly suited for the expression of antibody fragments. Such fragments are localised intracellularly or within the periplasma. Insoluble periplasmic proteins can be extracted and refolded to form active proteins according to methods known to those skilled in the art, see Sanchez et al (1999) J. Biotechnol. 72, 13-20 and Cupit P M et al (1999) Lett Appl Microbiol, 29, 273-277.

### 3. Pharmaceutical Compositions and Mode of Administration

Purified preparations of antibodies of the invention (particularly monoclonal preparations) as described supra, may be incorporated into pharmaceutical compositions for use in the treatment of human diseases and disorders such as those outlined above. Typically such compositions further comprise a pharmaceutically acceptable (i.e. inert) carrier as known and called for by acceptable pharmaceutical practice, see e.g. Remingtons Pharmaceutical Sciences, 16th ed., (1980), Mack Publishing Co. Examples of such carriers include sterilised carrier such as saline, Ringers solution or dextrose solution, buffered with suitable buffers to a pH within a range of 5 to 8. Pharmaceutical compositions for injection (e.g. by intravenous, intraperitoneal, intradermal, subcutaneous, intramuscular or intraportal) or continuous infusion are suitably free of visible particulate matter and may comprise from 1 mg to 10 g of therapeutic antibody, typically between 5 mg and 25 mg of antibody. Methods for the preparation of such pharmaceutical compositions are well known to those skilled in the art. In one embodiment, pharmaceutical compositions comprise from 1 mg to 10 g of therapeutic antibodies of the invention in unit dosage form, optionally together with instructions for use. Pharmaceutical compositions of the invention may be lyophilised (freeze dried) for reconstitution prior to administration according to methods well known or apparent to those skilled in the art. Where embodiments of the invention comprise antibodies of the invention with an IgG1 isotype, a chelator of copper such as citrate (e.g. sodium citrate) or EDTA or histidine may be added to the pharmaceutical composition to reduce the degree of copper-mediated degradation of antibodies of this isotype, see EP0612251.

Effective doses and treatment regimes for administering the antibody of the invention are generally determined empirically and are dependent on factors such as the age, weight and health status of the patient and disease or disorder to be treated. Such factors are within the purview of the attending physician. Guidance in selecting appropriate doses may be found in e.g. Smith et al (1977) Antibodies in human diagnosis and therapy, Raven Press, New York but will in general be between 1 mg and 1 g. In one embodiment, the dosing regime for treating a human patient is 1 mg to 10 g of therapeutic antibody of the invention administered subcutaneously once per week or every two weeks, for example, 40 mg of antibody delivered subcutaneously in 0.8 ml, or by intravenous infusion every 1 or 2 months, for example, 210-700 mg of therapeutic antibody in 250 ml of volume, infused over a period of 2 hours. Furthermore, initial induction of therapy could comprise a larger dosage for the first administration (e.g., 80-160 mg subcutaneously) or more frequent administration (e.g., intravenous infusions at 0 weeks, 2 weeks, and 6 weeks, followed by maintenance once every 8 weeks). Compositions of the present invention may also be used prophylatically

### 4. Clinical Uses.

The present invention relates to antibodies which bind to and neutralize human IL-8 The present invention also concerns methods of preventing or treating diseases or disorders characterised by elevated or unbalanced level of human IL-8 particularly endometriosis, COPD, osteoarthritis, rheumatoid arthritis, inflammatory bowel disease, psoriasis, pneumococcal meningitis, transplant rejection, gout, cystic fibrosis, adult respiratory distress syndrome, sepsis, reperfusion injury, or cancer, with said antibodies, pharmaceutical compositions comprising said antibodies and methods of manufacture.

The present invention also relates to use of a neutralizing antibody in the manufacture of a medicament for the prevention or treatment of diseases or disorders characterised by elevated or unbalanced level of human IL-8, particularly endometriosis, COPD, pneumococcal meningitis, osteoarthritis, rheumatoid arthritis, inflammatory bowel disease, psoriasis, transplant rejection, gout, cystic fibrosis, adult respiratory distress syndrome, sepsis, reperfusion injury, and cancer. Cancer indications may include, but are not limited to, glioblastoma, malignant mesothelioma, metastatic melanoma, metastatic breast cancer (estrogen-receptor-negative), metastatic pancreatic cancer, androgen-independent prostate cancer, and ovarian cancer.

Furthermore, the present invention also concerns methods of preventing or treating diseases or disorders characterised by elevated or unbalanced levels of human IL-8 and human VEGF, particularly endometriosis, osteoarthritis, rheumatoid arthritis, inflammatory bowel disease, reperfusion injury, or cancer. Thus in one embodiment, the present invention relates to a method of administering an anti-VEGF inhibitor in combination with any one of the aforementioned antibodies of the present invention for preventing or treating diseases or disorders characterised by elevated or unbalanced levels of human IL-8 and human VEGF, particularly, but not limited to, endometriosis, osteoarthritis, rheumatoid arthritis, inflammatory bowel disease, reperfusion injury, or cancer. VEGF inhibition may be accomplished through inhibition of VEGF or VEGF receptors, either using small molecular weight compounds, or monoclonal antibodies such as Avastin, antibody fragments such as Lucentis, or domain antibodies.

Although the present invention has been described principally in relation to the treatment of human diseases or disorders, the present invention may also have applications in the treatment of similar diseases or disorders in non-human mammals.

The following are non-limiting examples of the present invention.

### SPECIFIC EMBODIMENTS

The Table below shows the polypeptide sequences of representative mAbs

| mAbs | Heavy Chain | Light Chain |
|---|---|---|
| A0L0 | SEQ ID NO: 37 | SEQ ID NO: 39 |
| A0L1 | SEQ ID NO: 37 | SEQ ID NO: 40 |
| A0L2 | SEQ ID NO: 37 | SEQ ID NO: 41 |
| A1L0 | SEQ ID NO: 38 | SEQ ID NO: 39 |
| A1L1 | SEQ ID NO: 38 | SEQ ID NO: 40 |
| A1L2 | SEQ ID NO: 38 | SEQ ID NO: 41 |
| HcLc | SEQ ID NO: 27 | SEQ ID NO: 28 |

### Example 1

### Generation of mouse monoclonal antibody 1C1.5E5

Generation of an IL-8 specific neutralizing mAb.

Female SJL mice were immunized with a pool of recombinant human chemokines (IL-8, Gro-α, -β, -γ, and ENA-78) at multiple sites several times in one month. Serum samples were collected and analyzed by ELISA. The best responder was further boosted with the immunogens one and three days prior to the hybridoma generation. The spleen was excised, splenocytes prepared and a PEG (polyethylene glycol)-induced somatic fusion was performed with mouse myeloma cells P3X63BCL2-13. Among the many hybridomas obtained, a cell line producing 1C1.5E5 was obtained.

### Example 2

### Binding of the mAb to Human IL-8 was Confirmed Via an Enzyme-Linked Immunosorbent Assay (ELISA)

Hyridoma supernatants were screened for their antibody binding activities to recombinant human IL-8, Groβ and ENA-78, immobilized on ELISA plates. Hybridomas that showed IL-8 specificity but not cross-reactivity to Groβ or ENA-78 were selected. They were further assessed for their ability to recognize guinea pig and cynomolgus monkey orthologues. Selected hybridomas were cloned by limiting dilution cloning. The antibody designated 1C1.5E5 was one of the antibodies specific for human IL-8 that were selected for further analysis. Following humanization, the antibody combinations designated A0L0, A0L1, A0L2, AIL0, AIL1, and AIL2 also were determined to retain the ability to bind human IL-8.

### Example 3

### Functional IL-8 Neutralization was Confirmed Using a Variety of Methods: Calcium Flux Assay Read on Fluorescent Imaging Plate Reader (FLIPR), and Human Neutrophil Activation (CD11B Surface Expression)

Functional studies, neutralization of IL-8 induced Ca2+ flux, were performed on a CHO-K1 (Chinese hamster ovary) cell line stably expressing hCXCR2 w/ Ga16 or U2OS cells transfected with BacMam encoding hCXCR2 and Ga16. Cells were plated and grown for 24 hours in 96 well, black wall, clear bottom plates (Packard View). On the day of assay, cells were loaded with Fluo-4-acetoxymethyl ester fluorescent indicator dye (Fluo-4 AM, from Molecular Probes) and incubated at 37° C. in KRH solution. Separately, a 3×EC80 conc. of IL-8 was incubated for 60 minutes with a dilution range of mAb stock conc. (1:10 to 1:10000000). Plates were placed onto FLIPR (Fluorometric Imaging Plate Reader, Molecular Devices, Sunnyvale, Calif.) for analysis as described previously (Sarau et al., 1999). After baseline fluorescence detection, co-incubated ligand and mAb dilution were added to cells in FLIPR. The percent of maximal IL-8 induced Ca2+ mobilization induced by an EC80 concentration of IL-8 against CXCR2 was determined after treatment of cells with each dilution of mAb. The IC50 was calculated as the dilution of test mAb that inhibits 50% of the maximal response induced by IL-8. This value was then converted to ug/ml of mAb that inhibits 50% of the maximal response induced by IL-8. The 1C1.5E5 mAb inhibition of calcium mobilization expressed as IC50 was 0.1 ug/ml. The humanized antibodies retained the ability to inhibit calcium mobilization, with similar IC50s (range of 0.03-0.15 ug/ml for A0L0, A0L1, AIL0)

Inhibition of purified human neutrophil activation via monitoring surface expression of CD11b. CD11b or Mac-1 mediates adhesion of cells to substrates, participates in aggregation and chemotaxis and is known to be up-regulated on the surface activated neutrophils (Molad, Y., J., et al., Clin. Immunol. Immunopathol. 1994: 71; 281-286). Briefly, non-activated human neutrophils were purified and ex vivo stimulated with either target chemokines (i.e. IL-8) or with chemokines pre-incubated with an IL-8-specific neutralizing mAb. Data are presented as percent activation set to the maximal CD11b surface expression due to IL-8 stimulation alone. Pre-incubation of IL-8 with the 1C1.5E5 mAb dose—dependently inhibited increased levels of surface expression of CD11b and thus indicates inhibition of neutrophil activation (% cells positive for CD11b: approx. 60%, 48%, 28%, 17%, and 20% at 0.02, 0.2, 2, 20, and 200 μg/ml of 1C1.5E5 respectively for neutrophils obtained from one individual. Trends were the same from neutrophils obtained from other individuals). The humanized antibody A0L1 was tested in the same assay, using neutrophils obtained from 3 different donors, and retained the inhibition of neutrophil activation observed using the 1C1.5E5 mAb.

### Example 4

### IL-8-Specific Neutralizing Mabs Association/Dissociation Values for Human IL-8

### Methods for Biacore Analysis of the Parental Mabs Using a Capture Ligand.

Protein A or rabbit anti mouse IgG-Fc (Biacore BR-1005-14) was immobilised on a CM5 (Biacore BR-1000-14) chip by primary amine coupling in accordance with the manufactures instructions. Supernatant or purified from parental mouse mAb was captured on the antimouse IgG-Fc surface whilst chimeric antibody was captured on the Protein A surface. After capture defined concentrations of IL8 are passed over the antibody captured surface, a separate capture event was used for each analyte injection. After each injection of analyte the surface was regenerated by injection of a mild acidic solution, which removes the captured antibody but does not significantly affect the capability of the Protein A or anti mouse IgG-Fc surface to perform another capture event. An injection of buffer was also passed over the antibody captured surface and this was used for double referencing. For the analysis of the parental supernatant, an additional injection of an irrelevant mAb supernatant was injected over the antibody captured surface to detect any non-specific binding due to supernatant components. The data were analysed using the analysis software inherent to the machine, using the 1:1 model of binding. The work was carried out on the Biacore T100 or A100.

The KD values in the following table for IL-8-specific antibodies 101 and its subclone 1C1.5E5 and subclones of 1C1.5E5 are values measured by surface plasmon resonance as substantially described as above.

| | Human IL-8 | | |
|---|---|---|---|
| mAb* | ka | kd | K_{D} |
| 1C1.5E5-1 | 3.22E+06 | 9.84E−05 | 3.06E−11 |
| 1C1.5E5-10 | 3.34E+06 | 2.45E−04 | 7.34E−11 |
| 1C1.5E5-11 | 5.66E+06 | 2.47E−04 | 4.37E−11 |
| 101.5E5-13 | 3.16E+06 | 2.43E−04 | 7.69E−11 |
| 1C1.5E5 | 4.05E+06 | 1.84E−04 | 4.54E−11 |

*The murine mAb of IgGl/κ class.

Method for Biacore Analysis of Humanized and Chimeric Mabs

Briefly the method for Biacore analysis was as follows, anti human IgG (Biacore BR-1008-39) was immobilised on a CM5 sensor chip via primary amine coupling. The humanised or chimeric antibodies were captured on this surface at between 440-500 resonance units (RU's). IL8 was then passed over the captured antibody surface at defined concentrations with regeneration using 3M MgCl₂ after each cycle. A buffer injection over the antibody captured surface was used for double referencing. The run was carried out at 25° C. using HBS-EP buffer, on A Biacore T100 machine. The data was analysed using the 1:1 model in the analysis software inherent to the machine. Table below details the results from two separate runs. Run 1 used IL8 concentrations of 64 nM, 16 nM, 4 nM, 1 nM, 0.25 nM, whilst Run 2 used II8 concentrations 32 nM, 16 nM, 8 nM, 4 nM, 2 nM, 1 nM, 0.5 nM, 0.25 nM.

| | ka (M⁻¹ · s⁻¹) | | kd (s⁻¹) | | | |
|---|---|---|---|---|---|---|
| | (on-rate) | | (off-rate) | | KD (pM) | |
| | Run 1 | Run 2 | Run 1 | Run 2 | Run 1 | Run 2 |
| A0L0 | 1.11e+7 | 9.5e+6 | 7.91e−4 | 7.72e−4 | 71.2 | 81.6 |
| A0L1 | 1.49e+7 | 1.56e+7 | 1.00e−3 | 1.06e−3 | 67.6 | 67.9 |
| A1L0 | 8.43e+6 | 7.87e+6 | 7.35e−4 | 7.50e−4 | 87.2 | 95.4 |
| HcLc | 2.75e+7 | 2.68e+7 | 5.06e−4 | 5.66e−4 | 18.4 | 21.1 |

### Example 5

### IL-8 Neutralization Reduces Endometriosis Lesion Size and Number

Ovariectomized nude mice were implanted with estradiol-releasing silastic capsules. Human proliferative phase endometrial tissue is obtained, and maintained overnight in the presence of estradiol and antibiotics. After the 24-hour culture period, tissues were washed in PBS and injected into mice intraperitoneally. Treatment with therapeutic mAb was initiated 10 days after tissue injection. The 1C1.5E5 mAb was dosed at 10 mg/kg two times per week. Animals were necropsied for signs of experimental endometriosis 30 days after tissue injection.

In two separate experiments, the 1C1.5E5 mAb caused almost complete regression of endometrial lesions. Experiment 1: 10 lesions in control mice (average size of 2.6 mm); 1 lesion in 1C1.5E5 mice (1.0 mm). Experiment 2: 4 lesions in control mice 1.1 mm); 0 lesions in 1C1.5E5 mice.

### Epitope Mapping

1C1.5E5 mAb was epitope mapped and found to bind within KTYSKPFHPKFI (SEQ ID NO: 31) in human IL-8. Thus in another embodiment, the present invention relates to an IL-8-specific antibody which binds within epitope of SEQ ID NO: 31 of human IL-8.

In one embodiment, an antibody of the present invention is an antibody which has the ability to block the binding the 1C1.5E5 monoclonal antibody to an antigen in an ELISA assay.

In another embodiment, an antibody of the present invention is an antibody which can compete with 1C1.5E5 for binding to an epitope within SEQ ID NO: 31 of human IL-8.

### Cloning of Hybridoma Variable Regions

### Variable Region Sequences

Total RNA was extracted from 1C1.5E5 hybridoma cells, heavy and light variable domain cDNA sequence was then generated by reverse transcription and polymerase chain reaction (RT-PCR). The forward primer for RT-PCR was a mixture of degenerate primers specific for murine immunoglobulin gene leader-sequences and the reverse primer was specific for the antibody constant regions, in this case isotype IgG1/κ. Primers were designed according to the strategy described by Jones and Bendig (Bio/Technology 9:88, 1991). RT-PCR was carried out in duplicate for both V-region sequences to enable subsequent verification of the correct V-region sequences. The V-region products generated by the RT-PCR were cloned (Invitrogen TA Cloning Kit) and sequence data obtained.

### Sequence Table

Polynucleotide sequences for heavy and light variable regions (SEQ ID NO: 1 and 2, respectively) for 1C1.5E5

SEQ ID NO: 1
CAGGTCCAACTGCAGCAGCCTGGGGTTGAGCTTGTGATGCCTGGGG
CTTCAGTGAAGCTGTCCTGCAAGGCTTCTGGCTACACCTTCACCAG
CTACTGGATGCACTGGGTGAAGCAGAGGCCTGGACAAGGCCTTGAG
TGGATCGGCGAGATTGATCCTTCTGATAGTAATACTAACTACAATC
AAAAGTTCAAGGGCAAGGCCACATTGACTTTAGACAAATCCTCCAG
CACAGCCTACATGCAGCTCACCAGCCTGACATCTGAGGACTCAGCG
GTCTATTACTGTGCAAGAGAACTACTGCATGCGGTCTATTGGGGCC
AAGGCACCACTCTCACAGTCTCCTCA
SEQ ID NO: 2
GACATCCAGATGACACAGTCTCCATCCTCACTGTCTGCATCTCTGG
GGGGCAAAGTCACCATCACTTGCACGGCAAGCCAAGACATTCACAA
ATATATATCTTGGTTCCAACATAAGCCTGGAAAAGGTCCTAGACTG
CTCATACATTACACATCTACATTACAGCCAGGCATCCCATCAAGGT
TCAGTGGGAGTGGGTCTGGGAGAGATTATTCCTTCAGCATCAGCAA
CCTGGAGCCTGAAGATGTTGCAACTTATTATTGTCTACAATATGAT
AATCTGTGGACGTTCGGTGGAGGCACCAAGCTGGATATCAAACGGG
CT

Polypeptide sequences for heavy and light variable regions (SEQ ID NO: 3 and 4, respectively) for 1C1.5E5. Complementarity Determining Regions (CDRs) are underlined.

SEQ ID NO: 3
QVQLQQPGVELVMPGASVKLSCKASGYTFTSYWMHWVKQRPGQGLE
WIGEIDPSDSNTNYNQKFKGKATLTLDKSSSTAYMQLTSLTSEDSA
VYYCARELLHAVYWGQGTTLTVSS
SEQ ID NO: 4
DIQMTQSPSSLSASLGGKVTITCTASQDIHKYISWFQHKPGKGPRL
LIHYTSTLQPGIPSRFSGSGSGRDYSFSISNLEPEDVATYYCLQYD
NLWTFGGGTKLDIKRA

Polypeptide sequences for heavy chain CDRs (SEQ ID NO: 5, 6, 7, respectively) for 1C1.5E5

SEQ ID NO: 5
SYWMH
SEQ ID NO: 6
EIDPSDSNTNYNQKFKG
SEQ ID NO: 7
ELLHAVY

Polypeptide sequences for light chain CDRs (SEQ ID NO: 8, 9, 10) for 1C1.5E5

SEQ ID NO: 8
TASQDIHKYIS
SEQ ID NO: 9
TSTLQP
SEQ ID NO: 10
LQYDNLWT

Polynucleotide sequences for heavy chain CDRs (SEQ ID NO: 11, 12, 13) for 1C1.5E5

SEQ ID NO: 11
AGCTACTGGATGCAC
SEQ ID NO: 12
GAGATTGATCCTTCTGATAGTAATACTAACTACAATCAAAAGTTCA
AGGGC
SEQ ID NO: 13
GAACTACTGCATGCGGTCTAT

Polynucleotide sequences for light chain CDRs (SEQ ID NO:14, 15, 16) for 1C1.5E5

SEQ ID NO: 14
ACGGCAAGCCAAGACATTCACAAATATATATCT
SEQ ID NO: 15
ACATCTACATTACAGCCA
SEQ ID NO: 16
CTACAATATGATAATCTGTGGACG

SEQ ID NOs: 17-26 relate to humanized 1C1.5E5 antibodies.

Polynucleotide sequence: A0 heavy chain variable
SEQ ID NO: 17
CAGGTGCAGCTGGTGCAGAGCGGCGCCGAGGTGAAGAAGCCCGGCGCC
AGCGTGAAAGTGAGCTGCAAGGCCAGCGGCTACACCTTCACCAGCTAC
TGGATGCACTGGGTCAGGCAGGCTCCCGGCCAGGGCCTGGAGTGGATG
GGCGAGATCGACCCCAGCGACAGCAACACCAACTACAACCAGAAGTTC
AAGGGCAGGGTGACCATGACCAGGGACACCAGCATCAGCACCGCCTAC
ATGGAACTGAGCAGGCTGAGGTCCGACGACACCGCCGTGTACTATTGC
GCCAGGGAACTCCTGCACGCCGTGTACTGGGGGCAGGGAACACTAGTG
ACCGTGTCCAGC
Polynucleotide sequence: A1 heavy chain variable
SEQ ID NO: 18
CAGGTGCAGCTGGTGCAGAGCGGCGCCGAGGTGAAGAAGCCCGGCGCC
AGCGTGAAAGTGAGCTGCAAGGCCAGCGGCTACACCTTCACCAGCTAC
TGGATGCACTGGGTCAGGCAGGCTCCCGGCCAGGGCCTGGAGTGGATG
GGCGAGATCGACCCCAGCGACAGCAACACCAACTACAACCAGAAGTTC
AAGGGCAAGGTGACCATGACCAGGGACACCAGCATCAGCACCGCCTAC
ATGGAACTGAGCAGGCTGAGGTCCGACGACACCGCCGTGTACTATTGC
GCCAGGGAACTCCTGCACGCCGTGTACTGGGGGCAGGGAACACTAGTG
ACCGTGTCCAGC
Polynucleotide sequence: L0 light chain variable
SEQ ID NO: 19
GACATCCAGATGACCCAGTCTCCCAGCAGCCTGAGCGCCAGCGTGGGC
GACAGGGTGACCATTACCTGCACCGCCAGCCAGGACATCCACAAGTAC
ATCTCCTGGTACCAGCAGAAGCCCGGCAAGGCCCCCAAGCTGCTGATC
TACTACACTAGCACCCTGCAGCCCGGCGTCCCTTCAAGGTTCAGCGGA
AGCGGCAGCGGCACCGACTTCACCTTCACCATCAGCAGCCTGCAGCCC
GAGGATATCGCCACCTACTACTGCCTGCAGTACGACAACCTCTGGACC
TTCGGCCAGGGCACCAAAGTGGAGATCAAGCGT
Polynucleotide sequence: L1 light chain variable
SEQ ID NO: 20
GACATCCAGATGACCCAGTCTCCCAGCAGCCTGAGCGCCAGCGTGGGC
GACAGGGTGACCATTACCTGCACCGCCAGCCAGGACATCCACAAGTAC
ATCTCCTGGTACCAGCAGAAGCCCGGCAAGGCCCCCAAGCTGCTGATC
CACTACACTAGCACCCTGCAGCCCGGCGTCCCTTCAAGGTTCAGCGGA
AGCGGCAGCGGCACCGACTTCACCTTCACCATCAGCAGCCTGCAGCCC
GAGGATATCGCCACCTACTACTGCCTGCAGTACGACAACCTCTGGACC
TTCGGCCAGGGCACCAAAGTGGAGATCAAGCGT
Polynucleotide sequence: L2 light chain variable
SEQ ID NO: 21
GACATCCAGATGACCCAGTCTCCCAGCAGCCTGAGCGCCAGCGTGGGC
GACAGGGTGACCATTACCTGCACCGCCAGCCAGGACATCCACAAGTAC
ATCTCCTGGTACCAGCAGAAGCCCGGCAAGGCCCCCAAGCTGCTGATC
CACTACACTAGCACCCTGCAGCCCGGCGTCCCTTCAAGGTTCAGCGGA
AGCGGCAGCGGCACCGACTACACCTTCACCATCAGCAGCCTGCAGCCC
GAGGATATCGCCACCTACTACTGCCTGCAGTACGACAACCTCTGGACC
TTCGGCCAGGGCACCAAAGTGGAGATCAAGCGT
Polypeptide sequence for A0 heavy chain variable
SEQ ID NO: 22
QVQLVQSGAEVKKPGASVKVSCKASGYTFTSYWMHWVRQAPGQGLEWM
GEIDPSDSNTNYNQKFKGRVTMTRDTSISTAYMELSRLRSDDTAVYYC
ARELLHAVYWGQGTLVTVSS
Polypeptide sequence for A1 heavy chain variable
SEQ ID NO: 23
QVQLVQSGAEVKKPGASVKVSCKASGYTFTSYWMHWVRQAPGQGLEWM
GEIDPSDSNTNYNQKFKGKVTMTRDTSISTAYMELSRLRSDDTAVYYC
ARELLHAVYWGQGTLVTVSS
Polypeptide sequence: L0 light chain variable
SEQ ID NO: 24
DIQMTQSPSSLSASVGDRVTITCTASQDIHKYISWYQQKPGKAPKLLI
YYTSTLQPGVPSRFSGSGSGTDFTFTISSLQPEDIATYYCLQYDNLWT
FGQGTKVEIKR
Polypeptide sequence: L1 light chain variable
SEQ ID NO: 25
DIQMTOSPSSLSASVGDRVTITCTASQDIHKYISWYQQKPGKAPKLLI
HYTSTLQPGVPSRFSGSGSGTDFTFTISSLQPEDIATYYCLQYDNLWT
FGQGTKVEIKR
Polypeptide sequence: L2 light chain variable
SEQ ID NO: 26
DIQMTOSPSSLSASVGDRVTITCTASQDIHKYISWYQQKPGKAPKLLI
HYTSTLQPGVPSRFSGSGSGTDYTFTISSLQPEDIATYYCLQYDNLWT
FGQGTKVEIKR
A chimera* polypeptide sequence (variable heavy
region + codon optimised IgG1)
SEQ ID NO: 27
QVQLQQPGVELVMPGASVKLSCKASGYTFTSYWMHWVKQRPGQGLEWI
GEIDPSDSNTNYNQKFKGKATLTLDKSSSTAYMQLTSLTSEDSAVYYC
ARELLHAVYWGQGTLVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNG
QRENNYKTTPPVLDSDGSFFLYSKLTVDKSRWQQGNVFSCSVMHEALH
NHYTQKSLSLSPGK.
Chimera* polypeptide sequence (variable light
region + codon optimised human cK)
SEQ ID NO: 28
DIQMTQSPSSLSASLGGKVTITCTASQDIHKYISWFQHKPGKGPRLLI
HYTSTLQPGIPSRFSGSGSGRDYSFSISNLEPEDVATYYCLQYDNLWT
FGGGTKLDIKRTVAAPSVFIFPPSDEQLKSGTASVVCLLNNFYPREAK
VQWKVDNALQSGNSQESVTEQDSKDSTYSLSSTLTLSKADYEKHKVYA
CEVTHQGLSSPVTKSFNRGEC.
A chimera* polynucleotide sequence (variable
heavy region + codon optimised IgG1)
SEQ ID NO: 29
CAGGTCCAACTGCAgCAGCCTGGGgTTGAGCTTGTGATGCCTGGGGCT
TCAGTGAAGCTGTCCTGCAAGGCTTCTGGCTACACCTTCACCAGCTAC
TGGATGCACTGGGTGAAGCAGAGGCCTGGACAAGGCCTTGAGTGGATC
GGCGAGATTGATCCTTCTGATAGTAATACTAACTACAATCAAAAGTTC
AAGGGCAAGGCCACATTGACTTTAGACAAATCCTCCAGCACAGCCTAC
ATGCAGCTCACCAGCCTGACATCTGAGGACTCAGCGGTCTATTACTGT
GCAAGAGAACTACTGCATGCGGTCTATTGGGGCCAAGGCACACTAGTC
ACAGTCTCCTCAGCCTCCACCAAGGGCCCATCGGTCTTCCCCCTGGCA
CCCTCCTCCAAGAGCACCTCTGGGGGCACAGCGGCCCTGGGCTGCCTG
GTCAAGGACTACTTCCCCGAACCGGTGACGGTGTCGTGGAACTCAGGC
GCCCTGACCAGCGGCGTGCACACCTTCCCGGCTGTCCTACAGTCCTCA
GGACTCTACTCCCTCAGCAGCGTGGTGACCGTGCCCTCCAGCAGCTTG
GGCACCCAGACCTACATCTGCAACGTGAATCACAAGCCCAGCAACACC
AAGGTGGACAAGAAAGTTGAGCCCAAATCTTGTGACAAAACTCACACA
TGCCCACCGTGCCCAGCACCTGAACTCCTGGGGGGACCGTCAGTCTTC
CTCTTCCCCCCAAAACCCAAGGACACCCTCATGATCTCCCGGACCCCT
GAGGTCACATGCGTGGTGGTGGACGTGAGCCACGAAGACCCTGAGGTC
AAGTTCAACTGGTACGTGGACGGCGTGGAGGTGCATAATGCCAAGACA
AAGCCGCGGGAGGAGCAGTACAACAGCACGTACCGTGTGGTCAGCGTC
CTCACCGTCCTGCACCAGGACTGGCTGAATGGCAAGGAGTACAAGTGC
AAGGTCTCCAACAAAGCCCTCCCAGCCCCCATCGAGAAAACCATCTCC
AAAGCCAAAGGGCAGCCCCGAGAACCACAGGTGTACACCCTGCCCCCA
TCCCGGGATGAGCTGACCAAGAACCAGGTCAGCCTGACCTGCCTGGTC
AAAGGCTTCTATCCCAGCGACATCGCCGTGGAGTGGGAGAGCAATGGG
CAGCCGGAGAACAACTACAAGACCACGCCTCCCGTGCTGGACTCCGAC
GGCTCCTTCTTCCTCTACAGCAAGCTCACCGTGGACAAGAGCAGGTGG
CAGCAGGGGAACGTCTTCTCATGCTCCGTGATGCATGAGGCTCTGCAC
AACCACTACACGCAGAAGAGCCTCTCCCTGTCTCCGGGTAAATGA
A chimera* polynucleotide sequence (variable
light region + codon optimised human cK)
SEQ ID NO: 30
GACATCCAGATGACACAGTCTCCATCCTCACTGTCTGCATCTCTGGGG
GGCAAAGTCACCATCACTTGCACGGCAAGCCAAGACATTCACAAATAT
ATATCTTGGTTCCAACATAAGCCTGGAAAAGGTCCTAGACTGCTCATA
CATTACACATCTACATTACAGCCAGGCATCCCATCAAGGTTCAGTGGG
AGTGGGTCTGGGAGAGATTATTCCTTCAGCATCAGCAACCTGGAGCCT
GAAGATGTTGCAACTTATTATTGTCTACAATATGATAATCTGTGGACG
TTCGGTGGAGGCACCAAGCTGGATATCAAACGTACGGTGGCTGCACCA
TCTGTCTTCATCTTCCCGCCATCTGATGAGCAGTTGAAATCTGGAACT
GCCTCTGTTGTGTGCCTGCTGAATAACTTCTATCCCAGAGAGGCCAAA
GTACAGTGGAAGGTGGACAACGCCCTCCAATCGGGTAACTCCCAGGAG
AGTGTCACAGAGCAGGACAGCAAGGACAGCACCTACAGCCTCAGCAGC
ACCCTGACGCTGAGCAAAGCAGACTACGAGAAACACAAAGTCTACGCC
TGCGAAGTCACCCATCAGGGCCTGAGCTCGCCCGTCACAAAGAGCTTC
AACAGGGGAGAGTGTTAG
Chimera* refers to chimeric antibody made from
murine 1C1.5E5 antibody
Polynucleotide sequence: A0 entire mature heavy
chain
SEQ ID NO: 32
CAGGTGCAGCTGGTGCAGAGCGGCGCCGAGGTGAAGAAGCCCGGCGCC
AGCGTGAAAGTGAGCTGCAAGGCCAGCGGCTACACCTTCACCAGCTAC
TGGATGCACTGGGTCAGGCAGGCTCCCGGCCAGGGCCTGGAGTGGATG
GGCGAGATCGACCCCAGCGACAGCAACACCAACTACAACCAGAAGTTC
AAGGGCAGGGTGACCATGACCAGGGACACCAGCATCAGCACCGCCTAC
ATGGAACTGAGCAGGCTGAGGTCCGACGACACCGCCGTGTACTATTGC
GCCAGGGAACTCCTGCACGCCGTGTACTGGGGGCAGGGAACACTAGTG
ACCGTGTCCAGCGCCAGCACCAAGGGCCCCAGCGTGTTCCCCCTGGCC
CCCAGCAGCAAGAGCACCAGCGGCGGCACAGCCGCCCTGGGCTGCCTG
GTGAAGGACTACTTCCCCGAACCGGTGACCGTGTCCTGGAACAGCGGA
GCCCTGACCAGCGGCGTGCACACCTTCCCCGCCGTGCTGCAGAGCAGC
GGCCTGTACAGCCTGAGCAGCGTGGTGACCGTGCCCAGCAGCAGCCTG
GGCACCCAGACCTACATCTGTAACGTGAACCACAAGCCCAGCAACACC
AAGGTGGACAAGAAGGTGGAGCCCAAGAGCTGTGACAAGACCCACACC
TGCCCCCCCTGCCCTGCCCCCGAGCTGCTGGGAGGCCCCAGCGTGTTC
CTGTTCCCCCCCAAGCCTAAGGACACCCTGATGATCAGCAGAACCCCC
GAGGTGACCTGTGTGGTGGTGGATGTGAGCCACGAGGACCCTGAGGTG
AAGTTCAACTGGTACGTGGACGGCGTGGAGGTGCACAATGCCAAGACC
AAGCCCAGGGAGGAGCAGTACAACAGCACCTACCGGGTGGTGTCCGTG
CTGACCGTGCTGCACCAGGATTGGCTGAACGGCAAGGAGTACAAGTGT
AAGGTGTCCAACAAGGCCCTGCCTGCCCCTATCGAGAAAACCATCAGC
AAGGCCAAGGGCCAGCCCAGAGAGCCCCAGGTGTACACCCTGCCCCCT
AGCAGAGATGAGCTGACCAAGAACCAGGTGTCCCTGACCTGCCTGGTG
AAGGGCTTCTACCCCAGCGACATCGCCGTGGAGTGGGAGAGCAACGGC
CAGCCCGAGAACAACTACAAGACCACCCCCCCTGTGCTGGACAGCGAT
GGCAGCTTCTTCCTGTACAGCAAGCTGACCGTGGACAAGAGCAGATGG
CAGCAGGGCAACGTGTTCAGCTGCTCCGTGATGCACGAGGCCCTGCAC
AATCACTACACCCAGAAGAGCCTGAGCCTGTCCCCTGGCAAGTGA
Polynucleotide sequence: A1 entire mature heavy
chain
SEQ ID NO: 33
CAGGTGCAGCTGGTGCAGAGCGGCGCCGAGGTGAAGAAGCCCGGCGCC
AGCGTGAAAGTGAGCTGCAAGGCCAGCGGCTACACCTTCACCAGCTAC
TGGATGCACTGGGTCAGGCAGGCTCCCGGCCAGGGCCTGGAGTGGATG
GGCGAGATCGACCCCAGCGACAGCAACACCAACTACAACCAGAAGTTC
AAGGGCAAGGTGACCATGACCAGGGACACCAGCATCAGCACCGCCTAC
ATGGAACTGAGCAGGCTGAGGTCCGACGACACCGCCGTGTACTATTGC
GCCAGGGAACTCCTGCACGCCGTGTACTGGGGGCAGGGAACACTAGTG
ACCGTGTCCAGCGCCAGCACCAAGGGCCCCAGCGTGTTCCCCCTGGCC
CCCAGCAGCAAGAGCACCAGCGGCGGCACAGCCGCCCTGGGCTGCCTG
GTGAAGGACTACTTCCCCGAACCGGTGACCGTGTCCTGGAACAGCGGA
GCCCTGACCAGCGGCGTGCACACCTTCCCCGCCGTGCTGCAGAGCAGC
GGCCTGTACAGCCTGAGCAGCGTGGTGACCGTGCCCAGCAGCAGCCTG
GGCACCCAGACCTACATCTGTAACGTGAACCACAAGCCCAGCAACACC
AAGGTGGACAAGAAGGTGGAGCCCAAGAGCTGTGACAAGACCCACACC
TGCCCCCCCTGCCCTGCCCCCGAGCTGCTGGGAGGCCCCAGCGTGTTC
CTGTTCCCCCCCAAGCCTAAGGACACCCTGATGATCAGCAGAACCCCC
GAGGTGACCTGTGTGGTGGTGGATGTGAGCCACGAGGACCCTGAGGTG
AAGTTCAACTGGTACGTGGACGGCGTGGAGGTGCACAATGCCAAGACC
AAGCCCAGGGAGGAGCAGTACAACAGCACCTACCGGGTGGTGTCCGTG
CTGACCGTGCTGCACCAGGATTGGCTGAACGGCAAGGAGTACAAGTGT
AAGGTGTCCAACAAGGCCCTGCCTGCCCCTATCGAGAAAACCATCAGC
AAGGCCAAGGGCCAGCCCAGAGAGCCCCAGGTGTACACCCTGCCCCCT
AGCAGAGATGAGCTGACCAAGAACCAGGTGTCCCTGACCTGCCTGGTG
AAGGGCTTCTACCCCAGCGACATCGCCGTGGAGTGGGAGAGCAACGGC
CAGCCCGAGAACAACTACAAGACCACCCCCCCTGTGCTGGACAGCGAT
GGCAGCTTCTTCCTGTACAGCAAGCTGACCGTGGACAAGAGCAGATGG
CAGCAGGGCAACGTGTTCAGCTGCTCCGTGATGCACGAGGCCCTGCAC
AATCACTACACCCAGAAGAGCCTGAGCCTGTCCCCTGGCAAGTGA
Polynucleotide sequence: L0 entire mature light
chain
SEQ ID NO: 34
GACATCCAGATGACCCAGTCTCCCAGCAGCCTGAGCGCCAGCGTGGGC
GACAGGGTGACCATTACCTGCACCGCCAGCCAGGACATCCACAAGTAC
ATCTCCTGGTACCAGCAGAAGCCCGGCAAGGCCCCCAAGCTGCTGATC
TACGACACTAGCACCCTGCAGCCCGGCGTCCCTTCAAGGTTCAGCGGA
AGCGGCAGCGGCACCGACTTCACCTTCACCATCAGCAGCCTGCAGCCC
GAGGATATCGCCACCTACTACTGCCTGCAGTACGACAACCTCTGGACC
TTCGGCCAGGGCACCAAAGTGGAGATCAAGCGTACGGTGGCCGCCCCC
AGCGTGTTCATCTTCCCCCCCAGCGATGAGCAGCTGAAGAGCGGCACC
GCCAGCGTGGTGTGTCTGCTGAACAACTTCTACCCCCGGGAGGCCAAG
GTGCAGTGGAAGGTGGACAATGCCCTGCAGAGCGGCAACAGCCAGGAG
AGCGTGACCGAGCAGGACAGCAAGGACTCCACCTACAGCCTGAGCAGC
ACCCTGACCCTGAGCAAGGCCGACTACGAGAAGCACAAGGTGTACGCC
TGTGAGGTGACCCACCAGGGCCTGTCCAGCCCCGTGACCAAGAGCTTC
AACCGGGGCGAGTGCTGA
Polynucleotide sequence: L1 entire mature light
chain
SEQ ID NO: 35
GACATCCAGATGACCCAGTCTCCCAGCAGCCTGAGCGCCAGCGTGGGC
GACAGGGTGACCATTACCTGCACCGCCAGCCAGGACATCCACAAGTAC
ATCTCCTGGTACCAGCAGAAGCCCGGCAAGGCCCCCAAGCTGCTGATC
CACTACACTAGCACCCTGCAGCCCGGCGTCCCTTCAAGGTTCAGCGGA
AGCGGCAGCGGCACCGACTTCACCTTCACCATCAGCAGCCTGCAGCCC
GAGGATATCGCCACCTACTACTGCCTGCAGTACGACAACCTCTGGACC
TTCGGCCAGGGCACCAAAGTGGAGATCAAGCGTACGGTGGCCGCCCCC
AGCGTGTTCATCTTCCCCCCCAGCGATGAGCAGCTGAAGAGCGGCACC
GCCAGCGTGGTGTGTCTGCTGAACAACTTCTACCCCCGGGAGGCCAAG
GTGCAGTGGAAGGTGGACAATGCCCTGCAGAGCGGCAACAGCCAGGAG
AGCGTGACCGAGCAGGACAGCAAGGACTCCACCTACAGCCTGAGCAGC
ACCCTGACCCTGAGCAAGGCCGACTACGAGAAGCACAAGGTGTACGCC
TGTGAGGTGACCCACCAGGGCCTGTCCAGCCCCGTGACCAAGAGCTTC
AACCGGGGCGAGTGCTGA
Polynucleotide sequence: L2 entire mature light
chain
SEQ ID NO: 36
GACATCCAGATGACCCAGTCTCCCAGCAGCCTGAGCGCCAGCGTGGGC
GACAGGGTGACCATTACCTGCACCGCCAGCCAGGACATCCACAAGTAC
ATCTCCTGGTACCAGCAGAAGCCCGGCAAGGCCCCCAAGCTGCTGATC
CACTACACTAGCACCCTGCAGCCCGGCGTCCCTTCAAGGTTCAGCGGA
AGCGGCAGCGGCACCGACTACACCTTCACCATCAGCAGCCTGCAGCCC
GAGGATATCGCCACCTACTACTGCCTGCAGTACGACAACCTCTGGACC
TTCGGCCAGGGCACCAAAGTGGAGATCAAGCGTACGGTGGCCGCCCCC
AGCGTGTTCATCTTCCCCCCCAGCGATGAGCAGCTGAAGAGCGGCACC
GCCAGCGTGGTGTGTCTGCTGAACAACTTCTACCCCCGGGAGGCCAAG
GTGCAGTGGAAGGTGGACAATGCCCTGCAGAGCGGCAACAGCCAGGAG
AGCGTGACCGAGCAGGACAGCAAGGACTCCACCTACAGCCTGAGCAGC
ACCCTGACCCTGAGCAAGGCCGACTACGAGAAGCACAAGGTGTACGCC
TGTGAGGTGACCCACCAGGGCCTGTCCAGCCCCGTGACCAAGAGCTTC
AACCGGGGCGAGTGCTGA
Polypeptide sequence for A0 entire mature heavy
chain
SEQ ID NO: 37
QVQLVQSGAEVKKPGASVKVSCKASGYTFTSYWMHWVRQAPGQGLEWM
GEIDPSDSNTNYNQKFKGRVTMTRDTSISTAYMELSRLRSDDTAVYYC
ARELLHAVYWGQGTLVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPGK.
Polypeptide sequence for A1 entire mature heavy
chain
SEQ ID NO: 38
QVQLVQSGAEVKKPGASVKVSCKASGYTFTSYWMHWVRQAPGQGLEWM
GEIDPSDSNTNYNQKFKGKVTMTRDTSISTAYMELSRLRSDDTAVYYC
ARELLHAVYWGQGTLVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPGK.
Polypeptide sequence for L0 entire mature light
chain
SEQ ID NO: 39
DIQMTQSPSSLSASVGDRVTITCTASQDIHKYISWYQQKPGKAPKLLI
YDTSTLQPGVPSRFSGSGSGTDFTFTISSLQPEDIATYYCLQYDNLWT
FGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYA
CEVTHQGLSSPVTKSFNRGEC.
Polypeptide sequence for L1 entire mature light
chain
SEQ ID NO: 40
DIQMTQSPSSLSASVGDRVTITCTASQDIHKYISWYQQKPGKAPKLLI
HYTSTLQPGVPSRFSGSGSGTDFTFTISSLQPEDIATYYCLQYDNLWT
FGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYA
CEVTHQGLSSPVTKSFNRGEC.
Polypeptide sequence for L2 entire mature light
chain
SEQ ID NO: 41
DIQMTQSPSSLSASVGDRVTITCTASQDIHKYISWYQQKPGKAPKLLI
HYTSTLQPGVPSRFSGSGSGTDYTFTISSLQPEDIATYYCLQYDNLWT
FGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYA
CEVTHQGLSSPVTKSFNRGEC
Polynucleotide sequence: L0′ light chain variable
SEQ ID NO: 42
GACATCCAGATGACCCAGTCTCCCAGCAGCCTGAGCGCCAGCGTGGGC
GACAGGGTGACCATTACCTGCACCGCCAGCCAGGACATCCACAAGTAC
ATCTCCTGGTACCAGCAGAAGCCCGGCAAGGCCCCCAAGCTGCTGATC
TACGACACTAGCACCCTGCAGCCCGGCGTCCCTTCAAGGTTCAGCGGA
AGCGGCAGCGGCACCGACTTCACCTTCACCATCAGCAGCCTGCAGCCC
GAGGATATCGCCACCTACTACTGCCTGCAGTACGACAACCTCTGGACC
TTCGGCCAGGGCACCAAAGTGGAGATCAAGCGT
(In one embodiment, L0′ light chain variable
SEQ ID NO: 42 can be used instead of L0 light
chain variable SEQ ID NO: 19)
Polypeptide sequence: L0′ light chain variable
SEQ ID NO: 43
DIQMTQSPSSLSASVGDRVTITCTASQDIHKYISWYQQKPGKAPKLLI
YDTSTLQPGVPSRFSGSGSGTDFTFTISSLQPEDIATYYCLQYDNLWT
FGQGTKVEIKR
(In one embodiment, L0′ light chain variable
SEQ ID NO: 43 can be used instead of L0 light
chain variable SEQ ID NO: 24)

1. (canceled)

2. An antibody comprising heavy and light chain variable regions comprising amino acid sequences as set forth in SEQ ID NO:3 and SEQ ID NO:4, respectively, or one or more conservative sequence modifications thereof.

3. An antibody comprising heavy and light chain variable regions comprising polypeptides which are at least 90%, 95%, 98% or 99% identical to the amino acid sequences as set forth in SEQ ID NO:3 and SEQ ID NO:4, respectively.

4-7. (canceled)

8. An antibody comprising CDR sequences of SEQ ID NOs: 5, 6, 7, 8, 9, and 10; or one or more of the CDR sequences can be conservative sequence modifications of the sequences as set forth in sequences SEQ ID NOs: 5, 6, 7, 8, 9, and 10; or one or more of the CDR sequences are at least 90%, 95%, 98% or 99% identical to the amino acid sequences as set forth in sequences SEQ ID NOs: 5, 6, 7, 8, 9, and 10.

9-14. (canceled)

15. A host cell which produces an antibody comprising CDR sequences of SEQ ID NOs: 5, 6, 7, 8, 9, and 10; or one or more of the CDR sequences can be conservative sequence modifications of the sequences as set forth in sequences SEQ ID NOs: 5, 6, 7, 8, 9, and 10; or one or more of the CDR sequences are at least 90%, 95%, 98% or 99% identical to the amino acid sequences as set forth in sequences SEQ ID NOs: 5, 6, 7, 8, 9, and 10.

16-25. (canceled)

26. An expression vector comprising a nucleotide sequence encoding a variable heavy or light chain of an antibody comprising the CDR sequences of SEQ ID NOs: 5, 6, and 7; or SEQ ID NOs: 8, 9, and 10, respectively.

27-33. (canceled)

34. A process for producing an antibody in a host cell, comprising the steps of:
(i) introducing into said host cell a first DNA sequence encoding at least the variable domain of the antibody heavy chain comprising CDR domains of SEQ ID NOs: 5, 6, and 7; and a second DNA sequence encoding at least the variable domain of the antibody light chain comprising CDR domains of SEQ ID NOs: 8, 9, and 10; and
(ii) expressing said first DNA sequence and said second DNA sequence so that said antibody heavy and light chains are produced in said host cell.

35-72. (canceled)
